(19) United States
(12) Patent Application Publication
(10) Pub. No.: US 2012/0309794 A1
(43) Pub. Date: Dec. 6, 2012

(54) COMPOUNDS INHIBITORS OF ENZYME LACTATE DEHYDROGENASE (LDH) AND PHARMACEUTICAL COMPOSITIONS CONTAINING THESE COMPOUNDS

(75) Inventors: Filippo Minutolo, Pisa (PI) (IT); Marco Macchia, Livorno (Ll) (IT); Carlotta Granchi, Pontedera (Pl) (IT); Sarabindu Roy, West Bengal (IN); Gino Giannaccini, Forte dei Marmi (LU) (IT); Antonio Lucacchini, Forte dei Marmi (LU) (IT)

(73) Assignee: UNIVERSITÀ DI PISA, Pisa (PI) (IT)

(21) Appl. No.: 13/508,473

(22) PCT Filed: Nov. 5, 2010
(86) PCT No.: PCT/EP10/06740

§ 371 Date: May 7, 2012

(30) Foreign Application Priority Data
Nov. 9, 2009 (IT) PI2009A000140

(51) Int. Cl. A61K 31/404; A61P 35/00; C07D 403/10; A61K 31/4192; A61K 31/41; A61K 31/426; A61K 31/4439; C07D 413/10; A61K 31/4245; A61K 31/4184; C07D 235/26; C07D 277/46; C07D 209/42; C07D 401/14

(52) U.S. Cl. 514/339; 548/492; 514/419; 548/255; 514/414; 548/253; 514/381; 546/268.4; 548/132; 514/364; 514/394; 548/309.4; 548/195; 514/371

(57) ABSTRACT

The present invention concerns compounds, some of which are novel, and their pharmaceutical applications. The compounds of the invention inhibit the enzyme lactate dehydrogenase (LDH) involved both in the metabolic process of hypoxic tumour cells, and in the process used by parasitic protozoa that cause malaria to obtain most of the energy they need.

### FIELD OF INVENTION

The present invention concerns compounds, some of which are novel, and their pharmaceutical applications. The compounds of the invention inhibit the enzyme lactate dehydrogenase (LDH) involved both in the metabolic process of hypoxic tumour cells, and in the process used by parasitic protozoa that cause malaria to obtain most of the energy they need.

### BACKGROUND OF INVENTION

As widely known, tumour growth is associated to dramatic changes occurring to the normal structure of the affected organs, and it causes morphological alterations such as the progressive increase of the mean distance between blood vessels and tumour cells. As a consequence, many tumours, in particular solid tumours, turn out to be scarcely oxygenated. Under this condition, which is defined as “hypoxia”, tumours are particularly aggressive and prompt to form metastases.

Furthermore, hypoxic tumours display a strong resistance against traditional therapeutic treatments such as radiotherapy and chemotherapy. Radio-resistance in hypoxic tumour is mainly due to the low tendency to develop oxygen-dependent cytotoxic radicals upon irradiation. Chemo-resistance may, instead, be mostly due to the limited blood supply carrying the drug, as well as to the low proliferation level shown by hypoxic tumours, whereas the majority of currently employed chemotherapeutic agents target rapidly proliferating cells.

Therefore, there is a continuously growing interest in the search for alternative strategies for the treatment of hypoxic tumours. In particular, there are several ongoing studies about the use of compounds able to interfere with the main mechanisms utilized by hypoxic tumours to support their growth and invasiveness. For example, a group of prodrugs takes advantage of the reducing environment present in hypoxic tumours for their bioactivation process. Some of these prodrugs recently reached clinical phase trials [Brown J M, Wilson W R, Nat. Rev. Cancer 2004, 4, 437-447; Patterson A V et al., Clin. Cancer Res. 2007, 13, 3922-3932; Duan J-X et al., J. Med. Chem. 2008, 51, 2412-2420]. One of these prodrugs is tirapazamine, a benzotriazine able to release cytotoxic radicals upon reductive bioactivation in hypoxic conditions. However, this prodrug has a reduced ability of penetration into the tumour mass. Other prodrugs of the same kind have so far been employed in the treatment of hypoxic tumours, but their results were not completely satisfactory.

One of the most interesting features of tumour cells is their elevated glycolytic activity, which is up to 200-fold greater than that found in healthy cells [Gatenby R A, Gillies R J, Nat. Rev. Cancer 2004, 4, 891-899; Vander Heiden, M. G.; Cantley, L. C.; Thompson, C. B. Science 2009, 324, 1029-1033]. This is mainly due to: 1) high local consumption of oxygen that causes a shortage of this element and, consequently, increases the levels of anaerobic glycolysis; 2) presence of a higher amount of a particular form of enzyme hexokinase bound to mitochondria, which generates an increase of glycolytic activity, regardless the real consumption of oxygen. This phenomenon was described for the first time by Otto Warburg and, for this reason, it is also known as the “Warburg Effect” [Warburg O. On the origin of cancer cells. Science 1956, 123, 309-314].

As known, glycolysis is a metabolic process where a glucose molecule is cleaved into two pyruvate molecules. This generates higher-energy molecules such as two ATP and two NADH molecules.

Glycolysis comprises ten reactions occurring in the cell cytoplasm, which are catalyzed by specific enzymes, such as hexokinase, phosphoglucoisomerase, aldolase, and pyruvate kinase. Overall, this is a catabolic process since complex and high-energy molecules are converted to lower-energy simpler molecules, with consequent production of energy.

Glycolysis may take place both under aerobic conditions (in the presence of oxygen), and under anaerobic conditions (in the absence of oxygen). In both cases, one mole of glucose generates two moles of ATP, two moles of NADH and two moles of pyruvate. In the presence of oxygen, the pyruvate molecules produced by glycolysis are carried into the mitochondrial matrix, where they are decarboxylated and introduced into the Krebs cycle, also known as the tricarboxylic acid cycle, and then eventually transformed into carbonic anhydride, water and energy by means of oxidative phosphorylation.

On the other hand, under anaerobic conditions the pyruvic acid molecules are reduced to lactic acid (or lactate). This reaction is catalyzed by enzyme lactate dehydrogenase (LDH).

The majority of invasive tumour phenotypes, including haematological tumours such as leukaemia, display a neat metabolic switch from oxidative phosphorylation to anaerobic glycolysis. This guarantees a sufficient supply of energy and anabolic nutrients from glucCse to tumour cells even under anaerobic conditions.

An increase of anaerobic glycolysis mainly causes: 1) an elevated consumption of glucose, due to the low efficiency of this metabolic process; 2) an extracellular acidosis, due to the large amount of lactic acid produced by this process.

This peculiar tumour cell metabolism has inspired the search for innovative therapeutic approaches against cancer, by using molecules able to selectively inhibit one of those enzymes involved in the glycolytic pathway [Kraemer, G.; Pouyssegur, J. Cancer Cell 2008, 13, 472-482]. In fact, inhibition of one of the steps involved in the glycolytic pathway should provoke a blockage of the process used by tumour cells to produce most of the energy they need to survive and invade healthy tissues [Scatena, R.; Bottoni, P.; Pontoglio, A.; Mastrototaro, L.; Giardina, B. Expert Opin. Investig. Drugs 2008, 17, 1533-1545; Sheng, H.; Niu, B.; Sun, H. Curr. Med. Chem. 2009, 16, 1561-1587; Sattler, U. G. A.; Hirschhaeuser, F.; Mueller-Klieser, W. F. Curr. Med. Chem. 2010, 17, 96-108; Tennant, D. A.; Durán, R. V.; Gottlieb, E. Nat. Rev. Cancer 2010, 10, 267-277.].

Lonidamine is one of those molecules widely studied since it can interfere with cancer cell glycolysis by inhibiting enzyme hexokinase (HK) [Price, G. S.; Page, R. L.; Riviere, J. E.; Cline, J. M.; Thrall, D. E. Cancer Chemother. Pharmacol. 1996, 38, 129-135.]. In particular, hexokinase catalyzes the phosphorylation reaction of intracellular glucose to produce glucose-6-phosphate by using one molecule of ATP. This is the first step of glycolysis and one of the three fundamental steps of the whole pathway, since once glucose is to phosphorylated to glucose-6-phosphate, it cannot get out of the cell anymore through the cell membrane and, moreover, it becomes highly unstable and quickly liable to the subsequent catabolic sequence. However, Lonidamine also shows important side effects, such as pancreatic and hepatic toxicity.

Another widely studied hexokinase inhibitor is 2-deoxyglucose (2-DG). However, a scarce efficacy of 2-DG in the treatment of hypoxic tumours was recently reported. [Maher, J. C.; Wangpaichitr, M.; Savaraj, N.; Kurtoglu, M.; Lampidis, T. J. Mol. Cancer Ther. 2007, 6, 732-741]. Another HK-inhibitor is 3-bromopyruvate, but as of yet there are no available data about the clinical trials involving this compound [Ko, Y. H.; Smith, B. L.; Wang, Y.; et al. Biochem. Biophys. Res. Commun. 2004, 324, 269-275].

Dichloroacetate (DCA) is another molecules studied for its ability to interfere with the glycolytic process. DCA is an inhibitor of enzyme pyruvate dehydrogenase kinase (PDK), and it has currently reached clinical trials [Bonnet, S.; Archer, S. L.; Allalunis-Tumer, J.; et al. Cancer Cell 2007, 11, 37-51]. Lactate dehydrogenase (LDH) is one of the key enzymes involved in the peculiar glucose metabolism of cancer cells. As mentioned before, this enzyme catalyzes the reduction of pyruvate to lactate. In humans LDH (hLDH) is a tetrameric enzyme, which can exist in five predominant different isoforms (hLDH1-5), most of which are localized in cell cytosol. This tetrameric enzyme generally consists of two types of monomeric subunits, namely, LDH-A (or LDH-M from “muscle”) and LDH-B (or LDH-H, from “heart”), whose various combinations give rise to the following five tetrameric isoforms: hLDH1: LDH-B₄, hLDH2: LDH-AB₃, hLDH3: LDH-A₂B₂, hLDH4: LDH-A₃B and hLDH5: LDH-A₄. Among these isoforms, hLDH1 is mostly present in the heart, whereas hLDH5 is predominantly present in the liver and skeletal muscles.

Isoform hLDH5 of this enzyme, containing exclusively the LDH-A subunit, is overexpressed in highly invasive hypoxic tumours and it is clearly associated to hypoxia inducible factor 1 alpha (HIF-1α). Therefore, serum and plasma levels of hLDH5 are often utilized as tumour markers. These levels are not necessarily correlated to unspecific cell damage, but they may also be caused by an enzyme over-expression induced by malignant tumour phenotypes.

An amplification of this gene, measured as an increased production of subunit LDH-A, was verified in several cancer cell lines together with an over-production of glucose transporter GLUT1, following an induced oxygen deprivation [Sørensen B S et al., Radiother. Oncol. 2007, 83, 362-366]. Furthermore, the over-expression of LDH-A (as its fully functional tetrameric form, hLDH5) was found in many highly invasive hypoxic cancers [Koukorakis M I et al., Clin. Experim. Metast. 2005, 22, 25-30; Koukorakis M I et al., Cancer Sci. 2006, 97, 1056-1060] and this phenomenon could be clearly correlated to the intervention of HIF-1α [Kolev Y, Uetake H, Takagi Y, Sugihara K, Ann. Surg. Oncol. 2008, 15, 2336-2344]. Therefore, LDH-A was recently recognized as one of the most promising new targets for antitumour therapies, since its repression in invasive breast tumour cells was found to sensibly decrease cell invasiveness and tumour growth [Fantin V R, St-Pierre J, Leder P, Cancer Cell. 2006, 9, 425-434]. At the same time, the selective inhibition of this enzyme should not cause important side-effects in patients, since an hereditary deficiency of LDH-A found in some persons only produces myopathy after intense anaerobic exercise, whereas it does not give rise to any particular symptom under ordinary circumstances [Kanno T, Sudo K, Maekawa M, et al., Clin. Chim. Acta 1988, 173, 89-98].

Some examples of LDH-inhibition that produced an antitumour effect in cancer cell lines or tumours were reported in: P493 human lymphoma cells and xenografts [Le A, et al. Proc. Natl. Acad. Sci. U.S.A. 2010, 107, 2037-2042]; HepG2 and PLC/PRF/5 hepatocelilular carcinoma cells [Fiume L, et al. Pharmacology 2010, 86 (3), 157-162]; GS-2 glioblastoma, MDA-MB-231 breast cancer cells and murine xenografts [Ward C S, et al. Cancer Res. 2010, 70(4), 1296-1305; Mazzio E, Soliman K. WO2006017494]; taxol-resistant MDA-MD-435 human breast cancer cells [Zhou M, et al. Molecular Cancer 2010, 9, 33]; Dalton's lymphoma in murine models [Koiri R K, et al. Invest. New Drugs 2009, 27, 503-516; Pathak C, Vinayak M. Mol. Biol. Rep. 2005, 32, 191-196]; human cancer MCF (breast), KB (oral), KB-VIN (vincristine-resistant oral), SK-MEL-2 (melanoma), U87-MG (glioma), HCT-8 (colon), IA9 (ovarian), A549 (adenocarcinoma human alveolar cells) and PC-3 (prostate) cancer cell lines [Mishra L, et al. Indian J. Exp. Biol. 2004, 42(7), 660-666]; U87MG and AI72 glioma cells, primary glioma tumour cell culture “HTZ” [Baumann F, et al. Neuro-Oncology 2009, 11(4), 368-380]; Hereditary leiomyomatosis and renal cancer cell (HLRCC) syndrome, A549 adenocarcinoma human alveolar cells [Xie H, at al. Mol. Cancer Ther. 2009, 8(3), 626-635]; c-Myc-transformed Rat1 a fibroblasts, c-Myc-transformed human lymphoblastoid cells, and Burkitt lymphoma cells [Shim H, et al. Proc. Natl. Acad. Sci. U.S.A. 1997, 94, 6658-6663; Dang C, Shim H. WO9836774]; Burkitt lymphoma EB2 cells [Willsmore R L, Waring A J. IRCS Medical Science Library Compendium 1981, 9(11), 1003-1004]; colon adenocarcinoma HT29 and malignant glioma U118MG cells [Goerlach A, et al. Int. J. Oncol. 1995, 7(4), 831-839]; human glioma cell lines HS683, U373, U87 and U138, rat glioma cell line C6, SW-13 (adrenal), MCF-7 (breast), T47-D (breast), HeLa (cervical), SK-MEL-3 (melanoma), Cob 201 (colon) and BRW (cell line from a patient with a Primitive Neuroectodermal tumour) [Coyle T, at al. J. Neuro-Oncol. 1994, 19(1), 25-35].

Moreover, enzyme lactate dehydrogenase constitutes an interesting target for anti-malaric agents, since the parasitic protozoa causing malaria, during one phase of their infective cycle, utilize lactic fermentation to obtain most of their energy. Then, inhibitors of the LDH present in the etiological agent of malaria may be used as anti-malaric agents. In fact, some compounds were developed to block this infection by means of a selective inhibition of the plasmodial isoform of LDH, which, by the way, present a high level of homology when compared to human isoforms. [Turgut-Balik D et al., Biotechnol. Lett. 2004, 26, 1051-1055]. Most of the LDH-inhibitor so far developed were originally designed with the aim of producing new anti-malaric agents [Granchi C, Bertini S, Macchia M, Minutolo F, Curr. Med. Chem. 2010, 17, 672-697].

Another possible application of LDH-inhibitors is the treatment of tissue metaplasia and heterotopic ossification in idiopathic arthrofibrosis after total knee arthroplasty [Freeman T A, et al. Fibrogenesis Tissue Repair. 2010, 3, 17].

Furthermore, LDH-inhibitors may be used in cosmetic preparations, since they are able to stimulate the proliferation of cheratocytes and the biosynthesis of collagene in the skin [Bartolone J B, et al. U.S. Pat. No. 5,595,730 (1997)].

Compounds able to inhibit isoform C of lactate dehydrogenase may also be used as male contraceptives [Odet F, et al. Biol. Reprod. 2008, 79(1), 26-34; Yu Y, et al. Biochem. Pharmacol. 2001, 62, 81-89].

### SUMMARY OF THE INVENTION

It is therefore a feature of the present invention to provide compounds that are selective inhibitors of the LDH-A subunit of LDH enzymes.

It is another feature of the present invention to provide compounds for the treatment of tumor cells, in particular hypoxic tumour cells, through the selective inhibition of LDH enzymes.

It is another feature of the present invention to provide compounds for the treatment of tumor cells, in particular of cancer, in particular lymphoma, hepatocellular carcinoma, pancreatic cancer, brain cancer, breast cancer, lung cancer, colon cancer, cervical cancer, prostate cancer, kidney cancer, osteosarcoma, nasopharyngeal cancer, oral cancer, melanoma, ovarian carcinoma, with no relevant side effects for patients in treatment.

It is a particular feature of the present invention to provide compounds for the treatment of malaria with no relevant side effects for patients in treatment.

It is an additional feature of the present invention to provide compounds for the treatment of idiopathic arthrofibrosis with no relevant side effects for patients in treatment.

We have surprisingly found that compounds of formula I:

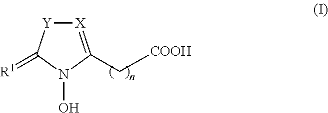

wherein:
n is selected from the group consisting of: 0, 1;
X is selected from the group consisting of: N, N⁺—O⁻, C—Z;
Y is selected from the group consisting of: S, O, C═R²;
Z is selected from the group consisting of: hydrogen, OR^{A}, NR^{A}R^{B}, halogen, cyano, nitro, alkoxy, aryloxy, heteroaryloxy, —C(O)C₁₋₆-alkyl, —C(O)phenyl, —C(O)benzyl, —C(O)C₅₋₆-heterocycle, —S—C₁₋₆-alkyl, —S-phenyl, —S-benzyl, —S—C₅₋₆-heterocycle, —S(O)C₁₋₆-alkyl, —S(O)phenyl, —S(O)benzyl, —S(O)₂C₅₋₆-heterocycle, —S(O)₂C₁₋₆-alkyl, —S(O)₂phenyl, —S(O)₂benzyl, —S(O)₂C₅₋₆-heterocycle, —S(O)₂NR^{A}R^{B}, C₁₋₆-alkyl, halo-C₁₋₆-alkyl, dihalo-C₁₋₆-alkyl, trihalo-C₁₋₆-alkyl, C₂₋₆-alkenyl, C₂₋₆-alkynyl, C₃₋₈-cycloalkyl, C₃₋₈-cycloalkyl-C₁₋₆-alkyl, phenyl, benzyl, and C₅₋₆-heterocycle;
R¹ is selected from:

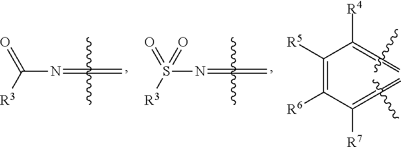

R² is selected, together with R¹, from:

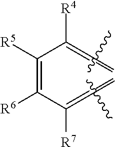

R³ is selected from the group consisting of: hydrogen, C₁₋₄-alkyl, halo-C₁₋₄-alkyl, dihalo-C₁₋₄-alkyl, trihalo-C₁₋₄-alkyl, C₂₋₆-alkenyl, C₂₋₄-alkynyl, C₃₋₆-cycloalkyl, C₃₋₆-cycloalkyl-C₁₋₂-alkyl, phenyl, benzyl, and C₅₋₆-heterocycle;
R⁴, R⁵, R⁶, R⁷ are independently selected from the group consisting of: hydrogen, OR^{A}, NR^{A}R^{B}, —C(O)R^{A}, —C(O)OR^{A}, —C(O)NR^{A}R^{B}, halogen, cyano, nitro, alkoxy, aryloxy, heteroaryloxy, —C(O)C₁₋₆-alkyl, —C(O)phenyl, —C(O)benzyl, —C(O)C₅₋₆-heterocycle, —S—C₁₋₆-alkyl, —S-phenyl, —S-benzyl, —S—C₅₋₆-heterocycle, —S(O)C₁₋₆-alkyl, —S(O)phenyl, —S(O)benzyl, —S(O)C₅₋₆-heterocycle, —S(O)₂C₁₋₆-alkyl, —S(O)₂phenyl, —S(O)₂benzyl, —S(O)₂C₅₋₆-heterocycle, —S(O)₂NR^{A}R^{B}, C₁₋₆-alkyl, halo-C₁₋₆-alkyl, dihalo-C₁₋₆-alkyl, trihalo-C₁₋₆-alkyl, C₂₋₆-alkenyl, C₂₋₆-alkynyl, C₃₋₈-cycloalkyl, C₃₋₈-cycloalkyl-C₁₋₆-alkyl, phenyl, benzyl, naphthyl, and C₅₋₆-heterocycle;
   wherein the phenyl, benzyl, naphthyl and C₅₋₆ heterocycle of the R³, R⁴, R⁵, R⁶, R⁷, R^{A} or R^{B} group may optionally be substituted with 1 to 3 groups independently selected from OR^{C} wherein two OR^{C} groups may concur into forming a cycle, NR^{C}R^{D}, —C(O)R^{c}, —C(O)OR^{c}, C₁₋₄-alkyl-OR^{c}, C₁₋₄-alkyl-C(O)OR^{c}, —C(O)NR^{C}R^{D}, —S(O)₂NR^{C}R^{D}, —S(O)₂C₁₋₆-alkyl, halogen, cyano, nitro, C₁₋₄-alkyl, halo-C₁₋₄-alkyl, dihalo-C₁₋₄-alkyl, trihalo-C₁₋₄-alkyl, aryl or heteroaryl optionally substituted with C(O)OR^{C}; wherein any atom of the C₅-C₆ heterocycle of the R³, R⁴, R⁵, R⁶ and R⁷ group may be bound to an oxygen so to form an oxo or a a sulfoxo moiety; wherein any alkyl, alkenyl and alkynyl groups of the R^{A}, R^{B}, R⁴, R⁵, R⁶ or R⁷ may optionally be substituted with 1-3 groups independently selected from OR^{C}, NR^{C}R^{D}, halogen, cyano and nitro; wherein any carbon-bound hydrogen atom may be substituted with a fluorine atom;
   R^{A}, R^{B}, R^{C} and R^{D} being independently selected from the group consisting of: hydrogen, —C(O)C₁₋₆-alkyl, —C(O)phenyl, —C(O)benzyl, —C(O)C₅₋₆-heterocycle, —S(O)₂C₁₋₆alkyl, —S(O)₂phenyl, —S(O)₂benzyl, —S(O)₂C₅₋₆-heterocycle, C₁₋₆-alkyl, halo-C₁₋₆-alkyl, dihalo-C₁₋₆-alkyl, trihalo-C₁₋₆-alkyl, C₂₋₆-alkenyl, C₂₋₆-alkynyl, C₃₋₈-cycloalkyl, C₃₋₈-cycloalkyl-C₁₋₆-alkyl, phenyl, benzyl, and C₅₋₆-heterocycle;
   are selective inhibitors of the LDH-A subunit of LDH enzymes.

None of the compounds according to formula (I) is known to have anti-LDH activity.

Accordingly, there is provided compounds inhibitors of the LDH-A subunit of a LDH enzyme, particularly LDH5, of general formula (I) above.

In one embodiment, the compounds of formula (I) are selected from those of formula (Ia):

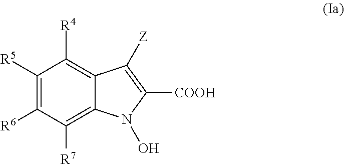

wherein Z, R⁴, R⁵, R⁶ and R⁷ are defined as under formula (I) above.

None of the compounds according to formula (Ia) is known in the art to possess biological activity that would render it suitable for use as a medicament.

Accordingly, there is provided compounds of formula (Ia) above for use a medicaments.

In a certain embodiment, there is provided novel compounds of formula (Ib)

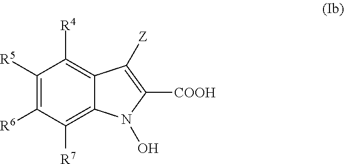

Wherein Z is either H or a C₁₋₆ alkyl; R⁴, R⁵, R⁶ and R⁷ are as defined under formula (I) above; and such that at least one of R⁴, R⁵, R⁶ and R⁷ is selected from the list of trihalo-C₁₋₄-alkyl, —S(O)₂NR^{A}R^{B}, phenyl, naphthyl or C₅₋₆ heterocycle optionally substituted with 1 to 3 groups independently selected from OR^{C}, NR^{C}R^{D}, —C(O)R^{c}, —C(O)OR^{c}, C₁₋₄-alkyl-OR^{c}, C₁₋₄-alkyl-C(O)OR^{c}, —C(O)NR^{C}R^{D}, —S(O)₂NR^{C}R^{D}, —S(O)₂C₁₋₆-alkyl, halogen, cyano, nitro, C₁₋₄-alkyl, halo-C₁₋₄-alkyl, dihalo-C₁₋₄-alkyl, trihalo-C₁₋₄-alkyl, aryl or heteroaryl optionally substituted with C(O)OR^{C}, and wherein R^{A}, R^{B}, R^{C} and R^{D} are as defined under formula (I) above.

In another embodiment there is provided a novel compound selected from the following list of (“list A”):
6-(3-carboxyphenyl)-1-hydroxy-1H-indol-2-carboxylic acid (Example 6);
5-(4-carboxy-1H-1,2,3-triazol-1-yl)-1-hydroxy-1H-indol-2-carboxylic acid (Example 12);
6-[4-(2-carboxyethyl)-1H-1,2,3-triazol-1-yl]-1-hydroxy-1H-indol-2-carboxylic acid (Example 14);
1-hydroxy-6-phenyl-4-trifluoromethyl-1H-indol-2-carboxylic acid (Example 20);
1-hydroxy-4-(4-phenyl-1H-1,2,3-triazol-1-yl)-1H-indol-2-carboxylic acid (Example 24);
1-hydroxy-6-[N-methyl-N-phenylsulfamoyl]-1H-indol-2-carboxylic acid (Example 26);
1-hydroxy-5-phenyl-1H-indol-2-carboxylic acid (Example 30);
1-hydroxy-6-(4-methoxyphenyl)-1H-indol-2-carboxylic acid (Example 31);
1-hydroxy-6-phenyl-1H-indol-2-carboxylic acid (Example 32);
1-hydroxy-6-(2H-tetrazol-5-yl)-1H-indol-2-carboxylic acid (Example 46);
5-[4-(2-carboxyethyl)phenyl]-1-hydroxy-1H-indol-2-carboxylic acid (Example 47);
4-[4-(3-carboxyphenyl)-1H-1,2,3-triazol-1-yl]-1-hydroxy-1H-indol-2-carboxylic acid (Example 48);
6-[4-(2-carboxyethyl)phenyl]-1-hydroxy-1H-indol-2-carboxylic acid (Example 49);
6-[4-(4-carboxyphenyl)-1H-1,2,3-triazol-1-yl]-1-hydroxy-1H-indol-2-carboxylic acid (Example 50);
5-(3-carboxyphenyl)-1-hydroxy-1H-indol-2-carboxylic acid (Example 56);
1-hydroxy-5,6-diphenyl-1H-indole-2-carboxylic acid (Example 57);
1-hydroxy-6-(N-methyl-N-p-tolylsulfamoyl)-1H-indole-2-carboxylic acid (Example 58);
1-hydroxy-6-(N-methyl-N-(4-(trifluoromethyl)phenyl)sulfamoyl)-1H-indole-2-carboxylic acid (Example 59);
6-(N-(4-fluorophenyl)-N-methylsulfamoyl)-1-hydroxy-1H-indole-2-carboxylic acid (Example 60);
6-(N-(4-chlorophenyl)-N-methylsulfamoyl)-1-hydroxy-1H-indole-2-carboxylic acid (Example 61);
5-(4-(3-carboxyphenyl)-1H-1,2,3-triazol-1-yl)-1-hydroxy-1H-indole-2-carboxylic acid (Example 62);
1-hydroxy-6-(4-(trifluoromethyl)phenyl)-1H-indole-2-carboxylic acid (Example 63);
6-(4-fluorophenyl)-1-hydroxy-1H-indole-2-carboxylic acid (Example 64);
5-(4-fluorophenyl)-1-hydroxy-1H-indole-2-carboxylic acid (Example 65);
1-hydroxy-5-(4-(trifluoromethyl)phenyl)-1H-indole-2-carboxylic acid (Example 66);
6-(benzo[d][1,3]dioxol-5-yl)-1-hydroxy-1H-indole-2-carboxylic acid (Example 67);
1-hydroxy-5-(4-methoxyphenyl)-1H-indole-2-carboxylic acid (Example 68);
6-(N-(2-chlorophenyl)-N-methylsulfamoyl)-1-hydroxy-1H-indole-2-carboxylic acid (Example 69);
6-(2,2-difluorobenzo[d][1,3]dioxol-5-yl)-1-hydroxy-1H-indole-2-carboxylic acid (Example 70);
5-(4-chlorophenyl)-1-hydroxy-1H-indole-2-carboxylic acid (Example 71);
6-(4-chlorophenyl)-1-hydroxy-1H-indole-2-carboxylic acid (Example 72);
1-hydroxy-6,7-diphenyl-4-(trifluoromethyl)-1H-indole-2-carboxylic acid (Example 73)
6-(N-butyl-N-phenylsulfamoyl)-1-hydroxy-1H-indole-2-carboxylic acid (Example 74);
6-(4-(N,N-dimethylsulfamoyl)phenyl)-1-hydroxy-1H-indole-2-carboxylic acid (Example 75);
6-(furan-3-yl)-1-hydroxy-1H-indole-2-carboxylic acid (Example 76);
1-hydroxy-6-(3-(trifluoromethoxy)phenyl)-1H-indole-2-carboxylic acid (Example 77);
6-(4-chlorophenyl)-1-hydroxy-4-(trifluoromethyl)-1H-indole-2-carboxylic acid (Example 78);
6-(biphenyl-4-yl)-1-hydroxy-1H-indole-2-carboxylic acid (Example 79);
1-hydroxy-3-methyl-6-phenyl-4-(trifluoromethyl)-1H-indole-2-carboxylic acid (Example 80);
1-hydroxy-6-(4-(trifluoromethoxy)phenyl)-1H-indole-2-carboxylic acid (Example 81);
1-hydroxy-6-(4-(N-methyl-N-phenylsulfamoyl)phenyl)-1H-indole-2-carboxylic acid (Example 82);
6-(4-chlorophenyl)-1-hydroxy-3-methyl-4-(trifluoromethyl)-1H-indole-2-carboxylic acid (Example 83);
1-hydroxy-6-(naphthalen-1-yl)-1H-indole-2-carboxylic acid (Example 84);
1-hydroxy-6-(naphthalen-2-yl)-1H-indole-2-carboxylic acid (Example 85);
6-(2,4-dichlorophenyl)-1-hydroxy-4-(trifluoromethyl)-1H-indole-2-carboxylic acid (Example 86);
6-(N-(3-chlorophenyl)-N-methylsulfamoyl)-1-hydroxy-1H-indole-2-carboxylic acid (Example 87);
1-hydroxy-5-(N-methyl-N-phenylsulfamoyl)-1H-indole-2-carboxylic acid (Example 88);

This invention is also directed to pharmaceutically acceptable salts, solvates, and to physiologically functional derivatives of:
compounds according to formulae (I), (Ia) or (Ib);
a compound selected from “list A” above.

Acid-derived pharmaceutically acceptable saltS not limitedly include hydrochlorides, hydrobromides, sulphates, nitrates, citrates, tartrates, acetates, phosphates, lactates, pyruvates, acetates, trifluoroacetates, succinates, perchlorates, fumarates, maleates, glycolates, salicylates, oxalates, oxalacetates, methansulfnonates, ethansulfonates, p-toluensolfates, formates, benzoates, malonates, naphatalen-2-sulphonates, isethionates, ascorbates, malates, phthalates, aspartates and glutamates, as well as arginine and lysine salts.

Base-derived pharmaceutically acceptable salts not limitedly include ammonium salts, alkaline metal salts, in particular sodium and potassium salts, alkaline earth metals salts, particularly calcium and magnesium salts, and organic base salts such as dicyclohkylamine, morpholine, thiomorpholine, piperidine, pyrrolidine, short chain mono-, di- or trialkylamines such as ethyl-, t-butyl, diethyl-, di-isopropyl, triethyl, tributyl or dimethylpropylamine, or short chain mono-, di- or trihydroxyalkylamines such as mono-, di-, or triethanolamine.

Other pharmaceutically acceptable salts can be internal salts, also known as zwitterions, whereby the molecule has regions of both negative and positive charge.

The skilled man in the art knows that any compound may form complexes together with the solvents in which it is dissolved into or precipitated or crystallised from. The complexes are known as solvates. For example, a complex with water is called a hydrate.

A “physiologically functional derivative” refers to any pharmaceutical acceptable derivative of a compound of the present invention, for example, an ester, an amide, or a carbamate, which upon administration to a mammal is capable of providing (directly or indirectly) a compound of the present invention or an active metabolite thereof. Such these derivatives are clear to those skilled in the art, without undue experimentation, and with reference to the teaching of Burger's Medicinal Chemistry And Drug Discovery, 5^{+h} Edition, Vol 1: Principles and Practice, which is incorporated herein by reference to the extent that it teaches physiologically functional derivatives.

Physiologically functional derivatives can also be obtained by conjugation of the molecule to carbohydrates [Gynther M, Ropponen J, Laine K, et al. J. Med. Chem. 2009, 52, 3348-3353; Lin Y-S Tungpradit R, Sinchaikul S, et at. J. Med. Chem. 2008, 51, 7428-7441; Thorson J S, Timmons S C, WO2010014814], amino acids or peptides [Singh S, Dash A K, Crit. Rev. Ther. Drug Carr. Syst. 2009, 26, 333-372; Hu Z, Jiang X, Albright C F, et al., Bioorg. Med. Chem. Lett. 2010, 20, 853-856.], and carriers that enhance the pharmacodynamic and pharmacokinetic properties of the compounds of interest.

In pharmaceutically acceptable esters, amides or carbamates, an appropriate group, for example a carboxyl group, is converted into an ester or amide with a C₁₋₆ alkyl group, a phenyl, a benzyl group, a C₅₋₈ heterocycle or an aminoacid.

In pharmaceutically acceptable esters, an appropriate group, for example an hydroxyl group, is converted into an ester with a a C₁₋₆ alkyl group, a phenyl, a benzyl group, a C₅₋₈ heterocycle or an aminoacid.

In pharmaceutically acceptable amides or carbamates, an appropriate group, for example an amine, is converted into an amide or a carbamate with a C₁₋₆ alkyl group, a phenyl, a benzyl group, a C₅₋₈ heterocycle or an aminoacid.

Accordingly, there is provided compounds of formula II, which are prodrugs of compounds of formula (I).

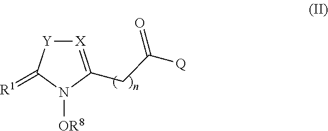

Wherein Q is OR^{E}, SR^{E} or NR^{E}R^{F} where R^{E} and R^{F}′ are independently selected from the group consisting of: hydrogen, —C(O)C₁₋₆-alkyl, —C(O)phenyl, —C(O)benzyl, —C(O)C₅₋₆-heterocycle, —S(O)₂C₁₋₆-alkyl, —S(O)₂phenyl, —S(O)₂benzyl, —S(O)₂C₅₋₆-heterocycle, C₁₋₆-alkyl, halo-C₁₋₆-alkyl, dihalo-C₁₋₆-alkyl, trihalo-C₁₋₆-alkyl, C₂₋₆-alkenyl, C₂₋₆-alkynyl, C₃₋₈-cycloalkyl, C₃₋₈-cycloalkyl-C₁₋₆-alkyl, phenyl, benzyl, C₅₋₆-heterocycle, an L- or a D-sugar, a deoxysugar, a dideoxysugar, a glucose epimer, an (un)substituted sugar, a uronic acid or an oligosaccharide; R⁸ is hydrogen, —C(O)C₁₋₆-alkyl, —C(O)phenyl, —C(O)benzyl, —C(O)C₅₋₆-heterocycle, trialkyl-silyl, dialkylaryl-silyl, C₁₋₄-alkyl, halo-C₁₋₄-alkyl, dialo-C₁₋₄-alkyl, trialo-C₁₋₄-alkyl, C₂₋₆-alkenyl, C₂₋₄-alkenyl, C₃₋₆-cycloalkyl, C₃₋₆-cycloalkyl-C₁₋₂-alkyl, phenyl, benzyl, C₅₋₆-heterocycle, an L- or a D-sugar, a deoxysugar, a dideoxysugar, a glucose epimer, an (un)substituted sugar, a uronic acid or an oligosaccharide and wherein R¹, n, Y and X are as defined under formula (I), (Ia) or (Ib).

It will be clear to the skilled man in the art that compounds of formula (III) below may be transformed, under reducing environment such as that of hypoxic tumours, into compounds of formula (II) or (I) upon administration to a mammal, because of the intermediate bioreductive transformation of the nitro-group to hydroxylamine [Brown J M, Wilson W R, Nat. Rev. Cancer 2004, 4, 437-447; Chen Y, Hu L, Med. Res. Rev. 2009, 29, 29-64] and subsequent condensation with the adjacent carbonyl portion.

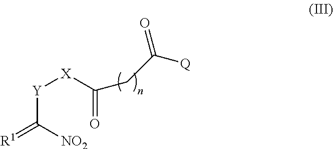

Wherein R¹, Y, X and Q are as defined under formula (II).

Accordingly, this invention is also directed to compounds of formula (III) above, which are prodrugs to compounds of formulae (II) and/or (I).

In the light of the biological activity of compounds of formula (I) against the LDH-A subunit of LDH enzymes, and in particular LDH5, any compound of the invention may be used for the cure of diseases associated with inhibition of that enzyme. In particular, these diseases can be selected from the list of cancer, particularly lymphoma, hepatocellular carcinoma, pancreatic cancer, brain cancer, breast cancer, lung cancer, colon cancer, cervical cancer, prostate cancer, kidney cancer, osteosarcoma, nasopharyngeal cancer, oral cancer, melanoma, ovarian carcinoma; malaria; idiopathic arthrofibrosis.

In some embodiments, there is provided pharmaceutical compositions which may contain:
one or more compounds of formulae (I), (Ia), (Ib), (II) and/or (III); or
one or more compounds selected from “list A” above and/or one or more of their respective prodrugs under formulae (II) or (III).

The pharmaceutical compositions of the invention comprise a pharmaceutically acceptable carrier and/or a pharmaceutically acceptable auxiliary substance. The pharmaceutical preparations can be administered orally, e.g. in the form of tablets, coated tablets, dragées, hard and soft gelatine capsules, solutions, emulsions or suspensions. The administration can, however, also be effected rectally, e.g. in the form of suppositories, or parenterally, e.g. in the form of injection solutions.

The compounds of the invention can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are however usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a compound of the invention and a therapeutically inert carrier are also an object of the present invention, as is a process for their production, which comprises bringing one or more compounds of the invention and, if desired, one or more other therapeutically valuable substances into a galenical administration form together with one or more therapeutically inert carriers.

The dosage can vary within wide limits and will, of course, have to be adjusted to the individual requirements in each particular case. In the case of oral administration the dosage for adults can vary from about 0.01 mg to about 1000 mg per day of a compound of the invention. The daily dosage may be administered as single dose or in divided doses and, in addition, the upper limit can also be exceeded when this is found to be indicated.

In some embodiments, such pharmaceutical preparations, particularly those for the cure of cancer, may be administered in combination with other pharmaceutically active agents. The phrase “in combination”, as used herein, refers to agents that are simultaneously administered to a subject. It will be appreciated that two or more agents are considered to be administered “in combination” whenever a subject is simultaneously exposed to both (or more) of the agents. Each of the two or more agents may be administered according to a different schedule; it is not required that individual doses of different agents be administered at the same time, or in the same composition. Rather, so long as both (or more) agents remain in the subject's body, they are considered to be administered “in combination”.

Upon exposure to ionising radiations or non-ionising radiations, particularly those falling in the infrared-visibile-ultraviolet range, the compounds of the invention are susceptible of releasing reactive oxygen species (ROS), in particular oxygenated radicals or peroxygenated groups with cytotoxic activity [Epe B, Ballmaier D, Adam W, Grimm G N, Saha-Möller C R, Nucleic Acid Res. 1996, 24, 1625-1631; Hwang J-T, Greenberg M M, Fuchs T, Gates K S, Biochemistry 1999, 38, 14248-14255; Xu G, Chance M R, Chem. Rev. 2007, 107, 3514-3543; Bischoff P, Altmeyer A, Dumont F, Exp. Opin. Ther. Pat. 2009, 19, 643-662]. In the field of cancer treatment, this property confers radiosentising or photosensitising properties to the pharmaceutical compositions of the invention. Accordingly, some embodiments of this invention also encompass uses of the pharmaceutical compositions of the invention in combination with radiation or photodynamic therapy for the treatment of cancer.

In some embodiments, the compounds of the invention used in a pharmaceutical compositions may be marked so as at to render them suitable as diagnostic agents.

In particular, the marking may be effected by introduction of:
a radionuclide,
a fluorophore,
ferromagnetic element;
a combination thereof.

Terms not specifically defined herein should be given the meanings that would be given to them by one of skill in the art in light of the disclosure and the context. As used in the specification and appended claims, however, unless specified to the contrary, the following terms have the meaning indicated below.

The term “alkyl” encompasses all saturated hydrocarbons, be them linear or branched. Non limiting examples include methyl, ethyl, n-propyl, iso-propyl, n-butyl, iso-butyl, t-butyl, sec-butyl, pentyl, hexyl, heptyl, octyl, nonyl, and decyl. Amongst the linear alkyls, methyl, ethyl, n-propyl and n-butyl are preferred. The branched alkyls not limitedly include: t-butyl, i-butyl, 1-ethylpropyl, 1-ethylbutyl and 1-ethylpentyl.

The term “alkoxy” encompasses O-alkyl groups, wherein alkyl is intended as described above. Non limiting examples of alkoxy groups include methoxy, ethoxy, propoxy and butoxy.

The term “alkenyl” encompasses unsaturated hydrocarbons, be these linear or branched, containing at least one carbon-carbon double bond. Alkenyl groups may, for example, contain up to five carbon-carbon double to bonds. Non limiting examples of alkenyl groups include ethenyl, propenyl, butenyl, pentenyl, hexenyl, heptenyl, octenyl, nonenyl, decenyl and dodecenyl. Preferred alkenyl groups include ethenyl, 1-propenyl and 2-propenyl.

The term “alkynyl” ecompasses unsaturated hydrocarbons, be these linear or branched, containing at least one triple carbon-carbon bond. Alkynyl groups may, for example, contain up to five carbon-carbon triple bonds. Non limiting examples of alkynyl groups include ethynyl, propynyl, butynyl, pentynyl, hexynyl, heptynyl, octynyl, nonynyl, decynyl and dodecynyl. Preferred alkynyl groups include ethynyl, 1-propynyl and 2-propynyl.

The term “cycloalkyl” encompasses cyclic saturated hydrocarbons. Cycloalkyl groups may be either monocyclic or bicyclic. A bicyclic group may be fused or bridged. Non limiting examples of cycloalkyl groups include cyclopropyl, cyclobutyl and cyclopentyl. Other non limiting examples of monocyclic cycloalkyls are cyclohexyl, cycloheptyl and cyclooctyl. An example of a bicyclic cycloalkyl is bicyclo[2.2.1]-hept-1-yl. The cycloalkyl group is preferably monocyclic.

The term “aryl” encompasses aromatic carbocyclic moieties which may be monocyclic or bicyclic. Non limiting examples of aryl groups are phenyl and naphthyl. A naphthyl group may be linked either via its 1- or its 2-position. In a bicyclic aromatic group, one of the rings may be saturated. Non limiting examples of such rings include indanyl and tetrahydronaphtyl. More specifically, a “C₅₋₁₀ aryl” group encompasses monocyclic or bicyclic aromatic systems containing 5 to 10 carbon atoms. A particulary preferred C₅₋₁₀ aryl group is phenyl.

The terms “aryloxy” encompasses O-aryl groups wherein aryl is intended as described above. A non limiting example of an aryloxy group is the phenoxy group.

The term “halogen” encompasses fluoro, chloro, bromo and iodo. Fluoro, chloro and bromo are particularly preferred. In some embodiments, fluoro is most preferred whereas in other embodiments chloro and bromo are most preferred.

The term “haloalkyl” encompasses alkyl groups harbouring an halogen subsituent, wherein alkyl and halogen are intended as described above. Similarly, the term “dihaloalkyl” encompasses alkyl groups having two halogen subsituents and the term “trihaloalkyl” encompasses alkyl groups harbouring three halogen substituents. Non limiting examples of haloakyl groups not limitedly include fluoromethyl, chloromethyl, bromomethyl, fluoroethyl, fluoropropyl and fluorobutyl; non limiting examples of dihaloalkyl groups are difluoromethyl and difluoroethyl; non limiting examples of trihaloalkyl groups are trifluoromethyl and trifluoroethyl.

The term “heterocyle” ecompasses aromatic (“heteroaryl”) or non-aromatic (“heterocycloalkyl”) carbocyclic groups wherein one to four carbon atoms is/are replaced by one or more heteroatoms selected from the list of nitrogen, oxygen and sulphur. An heterocyclic group may be monocyclic or bicyclic. Within a bicyclic heterocylic group, one or more heteroatoms may be found on either rings or in one of the rings only. Wherein valence and stability permit, nitrogen-containing heterocyclic groups also encompass their respective N-oxides. Non limiting examples of monocyclic hetroacycloalkyl include aziridinyl, azetidinyl, pyrrolidinyl, imidazolidinyl, pirazolidinyl, piperidinyl, piperazinyl, tetrahydrofuranyl, tetrahydropyranyl, morpholinyl, thiomorpholinyl and azepanyl.

More specifically, the term “C₅₋₁₀-heterocycle” encompasses a group containing 5 to 10 carbon atoms part of a mono- or bicyclic ring system which can be aromatic (“heteroaryl”) or non-aromatic (“heterocycloalkyl”) wherein one to four carbon atoms is/are replaced by one or more heteroatoms selected from the list of nitrogen, oxygen and sulphur. More precisely, the term “C₅-heterocycle” encompasses 5-membered cyclic aromatic (“heteroaryl”) or non aromatic (“heterocycloalkyl”) groups containing one or more heteroatoms independently selected from the list of nitrogen, oxygen and sulphur, whereas the remaining atoms forming the 5-membered ring are carbon atoms. Non limiting examples of C₅-heterocyclic groups include furanyl, thienyl, pyrrolyl, imidazolyl, oxazolyl, thiazolyl and their respective partially or fully saturated analogues such as dihydrofuranyl and tetrahydrofuranyl.

Non limiting examples of bicyclic eterocyclic groups wherein one of the two rings is not aromatic include dihydrobenzofuranyl, indanyl, indolinyl, tetrahydroisoquinolyl, tetrahydroquinolyl and benzoazepanyl.

Non limiting examples of monocyclic heteroaryl groups include furanyl, thienyl, pyrrolyl, oxazolyl, thiazolyl, imidazolyl, oxadiazolyl, thiadiazolyl, pyridyl, triazolyl, triazinyl, pyridazyl, pyrimidinyl, isothiazolyl, isoxazolyl, pyrazinyl, pyrazolyl and pyrimidinyl; non limiting examples of bicyclic heteroaryl groups include quioxalinyl, quinazolinyl, pyridopyrazolinyl, benzoxazolyl, benzothienyl, benzoimidazolyl, naphthyridyl, quinolinyl, benzofuranyl, indolyl, benzothiazolyl, oxazolyl[4,5-b]pyridyl, pyridopyrimidinyl and isoquinolinyl.

Non limiting examples of preferred heterocyclic groups are piperidinyl, tetrahydrofuranyl, tetrahydropyranyl, pyridyl, pyrimidinyl and indolyl. Other preferred heterocyclic group include thienyl, thiazolyl, furanyl, pyrazolyl, pyrrolyl, and imidazolyl.

The term “cycloalkylalkyl” encompasses cycloalkyl-alkyl groups, wherein cycloalkyl and alkyl have the meaning above described, which are bound via the alkyl group.

The term “heteroaryloxy” encompasses O-heteroaryl groups, wherein heteroaryl is intended as described above. Non limiting examples of heteroaryloxy groups are furanyloxy, thienyloxy, pyridinoxy.

The term “heterocycloalkoxy” encompasses O-heterocycloalkyl groups wherein heterocycloalkyl is intended as described above. Non limting examples of heterocycloalkoxy groups are piperidinyloxy, tetrahydrofuranyloxy, tetrahydropyranyloxy.

Whenever a chiral carbon is present in a chemical structure, it is intended that all stereoisomers associated with that chiral carbon are encompassed by the structure.

Furthermore, the invention includes all optical isomers, i.e. diastereoisomers, diastereomeric mixtures, racemic mixtures, all their corresponding enantiomers and/or tautomers.

### EXAMPLES

Examples 1-96 below are non limiting examples falling within the scope of the invention.

### Examples 1-88

### Falling Under Formula (Ib)

| Ex. | n | X | Y | Z | R⁴ | R⁵ | R⁶ | R⁷ |
|---|---|---|---|---|---|---|---|---|
| 1 | 0 | C—Z | C═R² | H | H | H | H | H |
| 2 | 0 | C—Z | C═R² | H | Br | H | H | H |
| 3 | 0 | C—Z | C═R² | H | Cl | H | H | H |
| 4 | 0 | C—Z | C═R² | H | H | H | Br | H |
| 5 | 0 | C—Z | C═R² | H | CH₃ | H | H | H |
| 6 | 0 | C—Z | C═R² | H | H | H | 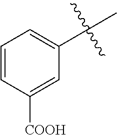 | H |
| 7 | 0 | C—Z | C═R² | H | H | H | 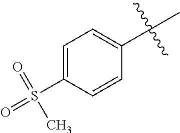 | H |
| 8 | 0 | C—Z | C═R² | H | H | 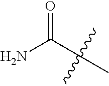 | H | H |
| 9 | 0 | C—Z | C═R² | H | H | F | H | H |
| 10 | 0 | C—Z | C═R² | CH₃ | H | H | H | H |
| 11 | 0 | C—Z | C═R² | C₂H₅ | H | H | H | H |
| 12 | 0 | C—Z | C═R² | H | H | 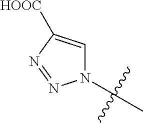 | H | H |
| 13 | 0 | C—Z | C═R² | H | H | H | 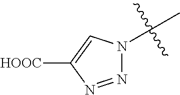 | H |
| 14 | 0 | C—Z | C═R² | H | H | H | 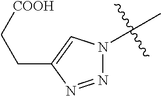 | H |
| 15 | 0 | C—Z | C═R² | H | H | H | 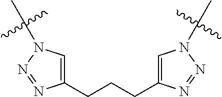 | H |
| 16 | 0 | C—Z | C═R² | H | H | H | 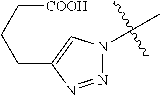 | H |
| 17 | 0 | C—Z | C═R² | H | H | H | 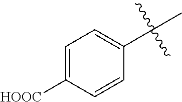 | H |
| 18 | 0 | C—Z | C═R² | H | H | H | 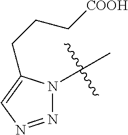 | H |
| 19 | 0 | C—Z | C═R² | H | H | 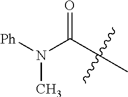 | H | H |
| 20 | 0 | C—Z | C═R² | H | CF₃ | H | Ph | H |
| 21 | 0 | C—Z | C═R² | H | H | 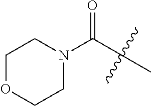 | H | H |
| 22 | 0 | C—Z | C═R² | H | 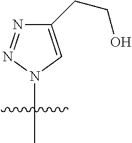 | H | H | H |
| 23 | 0 | C—Z | C═R² | H | H | 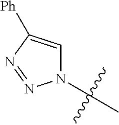 | H | H |
| 24 | 0 | C—Z | C═R² | H | 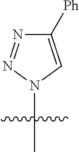 | H | H | H |
| 25 | 0 | C—Z | C═R² | H | H | H | 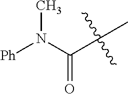 | H |
| 26 | 0 | C—Z | C═R² | H | H | H | 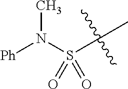 | H |
| 27 | 0 | C—Z | C═R² | H | H | H | 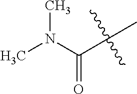 | H |
| 28 | 0 | C—Z | C═R² | H | H | H | 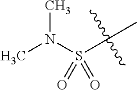 | H |
| 29 | 0 | C—Z | C═R² | H | H | H | 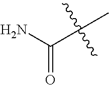 | H |
| 30 | 0 | C—Z | C═R² | H | H | Ph | H | H |
| 31 | 0 | C—Z | C═R² | H | H | H | 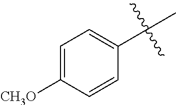 | H |
| 32 | 0 | C—Z | C═R² | H | H | H | Ph | H |
| 33 | 0 | C—Z | C═R² | H | H | COOH | H | H |
| 34 | 0 | C—Z | C═R² | H | H | H | F | H |
| 35 | 0 | C—Z | C═R² | H | H | CN | H | H |
| 36 | 0 | C—Z | C═R² | H | H | H | CN | H |
| 37 | 0 | C—Z | C═R² | H | F | H | H | H |
| 38 | 0 | C—Z | C═R² | H | CF₃ | H | H | H |
| 39 | 0 | C—Z | C═R² | H | H | F | Ph | H |
| 40 | 0 | C—Z | C═R² | H | Ph | H | H | H |
| 41 | 0 | C—Z | C═R² | H | 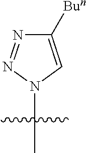 | H | H | H |
| 42 | 0 | C—Z | C═R² | H | H | H | 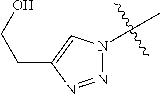 | H |
| 43 | 0 | C—Z | C═R² | H | H | 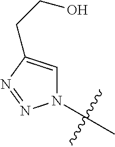 | H | H |
| 44 | 0 | C—Z | C═R² | H | H | 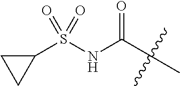 | H | H |
| 45 | 0 | C—Z | C═R² | H | H | H | 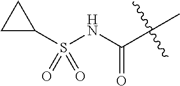 | H |
| 46 | 0 | C—Z | C═R² | H | H | H | 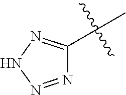 | H |
| 47 | 0 | C—Z | C═R² | H | H | 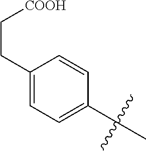 | H | H |
| 48 | 0 | C—Z | C═R² | H | 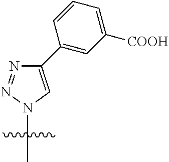 | H | H | H |
| 49 | 0 | C—Z | C═R² | H | H | H | 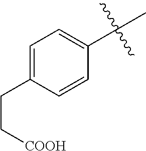 | H |
| 50 | 0 | C—Z | C═R² | H | H | H | 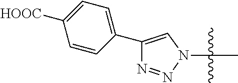 | H |
| 51 | 0 | C—Z | C═R² | H | H | 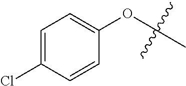 | H | H |
| 52 | 0 | C—Z | C═R² | H | H | 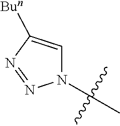 | H | H |
| 53 | 0 | C—Z | C═R² | H | H | H | 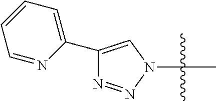 | H |
| 54 | 0 | C—Z | C═R² | H | H | H | 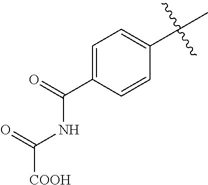 | H |
| 55 | 0 | C—Z | C═R² | H | H | H | 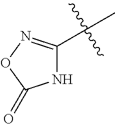 | H |
| 56 | 0 | C—Z | C═R² | H | H | 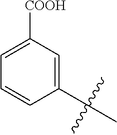 | H | H |
| 57 | 0 | C—Z | C═R² | H | H | Ph | Ph | H |
| 58 | 0 | C—Z | C═R² | H | H | H | 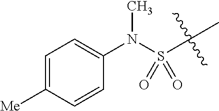 | H |
| 59 | 0 | C—Z | C═R² | H | H | H | 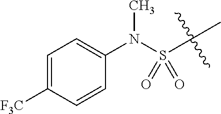 | H |
| 60 | 0 | C—Z | C═R² | H | H | H | 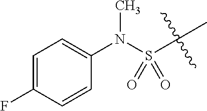 | H |
| 61 | 0 | C—Z | C═R² | H | H | H | 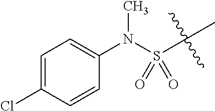 | H |
| 62 | 0 | C—Z | C═R² | H | H | 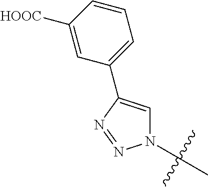 | H | H |
| 63 | 0 | C—Z | C═R² | H | H | H | 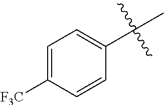 | H |
| 64 | 0 | C—Z | C═R² | H | H | H | 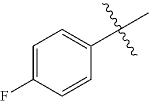 | H |
| 65 | 0 | C—Z | C═R² | H | H | 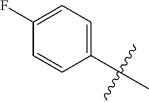 | H | H |
| 66 | 0 | C—Z | C═R² | H | H | 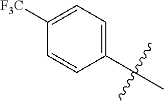 | H | H |
| 67 | 0 | C—Z | C═R² | H | H | H | 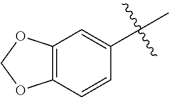 | H |
| 68 | 0 | C—Z | C═R² | H | H | 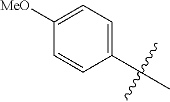 | H | H |
| 69 | 0 | C—Z | C═R² | H | H | H | 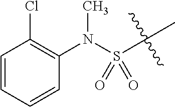 | H |
| 70 | 0 | C—Z | C═R² | H | H | H | 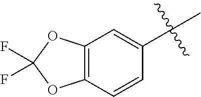 | H |
| 71 | 0 | C—Z | C═R² | H | H | 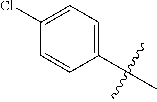 | H | H |
| 72 | 0 | C—Z | C═R² | H | H | H | 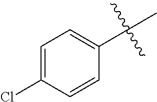 | H |
| 73 | 0 | C—Z | C═R² | H | CF₃ | H | Ph | Ph |
| 74 | 0 | C—Z | C═R² | H | H | H | 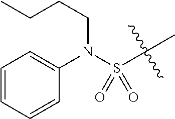 | H |
| 75 | 0 | C—Z | C═R² | H | H | H | 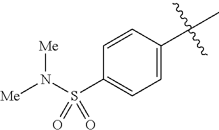 | H |
| 76 | 0 | C—Z | C═R² | H | H | H | 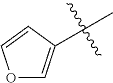 | H |
| 77 | 0 | C—Z | C═R² | H | H | H | 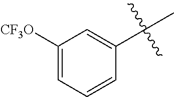 | H |
| 78 | 0 | C—Z | C═R² | H | CF₃ | H | 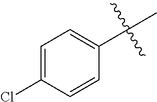 | H |
| 79 | 0 | C—Z | C═R² | H | H | H | 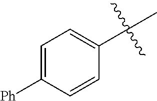 | H |
| 80 | 0 | C—Z | C═R² | CH₃ | CF₃ | H | Ph | H |
| 81 | 0 | C—Z | C═R² | H | H | H | 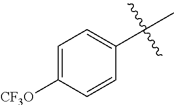 | H |
| 82 | 0 | C—Z | C═R² | H | H | H | 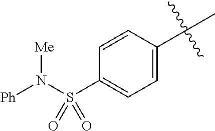 | H |
| 83 | 0 | C—Z | C═R² | CH₃ | CF₃ | H | 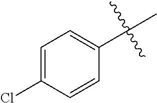 | H |
| 84 | 0 | C—Z | C═R² | H | H | H | 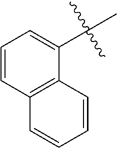 | H |
| 85 | 0 | C—Z | C═R² | H | H | H | 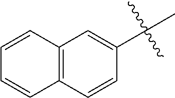 | H |
| 86 | 0 | C—Z | C═R² | H | CF₃ | H | 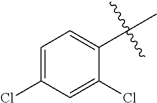 | H |
| 87 | 0 | C—Z | C═R² | H | H | H | 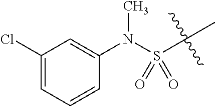 | H |
| 88 | 0 | C—Z | C═R² | H | H | 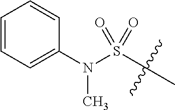 | H | H |

### Examples 89-92

### Falling Under Formula (I) Wherein R¹ and R² are

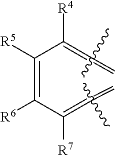

| Ex. | n | X | Y | Z | R⁴ | R⁵ | R⁶ | R⁷ |
|---|---|---|---|---|---|---|---|---|
| 89 | 0 | N | C═R² | — | H | H | H | H |
| 90 | 0 | N⁺—O⁻ | C═R² | — | H | H | H | H |
| 91 | 0 | N⁺—O⁻ | C═R² | — | H | H | Cl | H |
| 92 | 0 | N⁺—O⁻ | C═R² | — | H | H | Ph | H |

### Examples 93-96

### Falling Under Formula (I) Wherein R¹ is

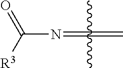

| Ex. | n | X | Y | Z | R³ | R⁴ | R⁵ | R⁶ | R⁷ |
|---|---|---|---|---|---|---|---|---|---|
| 93 | 1 | C—Z | S | H | Ph | — | — | — | — |
| 94 | 1 | C—Z | S | H | CH₃ | — | — | — | — |
| 95 | 1 | C—Z | S | H | 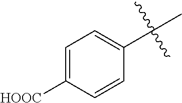 | — | — | — | — |
| 96 | 1 | C—Z | S | H | 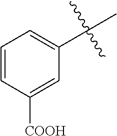 | — | — | — | — |

The IUPAC names of the above examples are listed below:
1-hydroxy-1H-indol-2-carboxylic acid (Example 1);
4-bromo-1-hydroxy-1H-indol-2-carboxylic acid (Example 2);
4-chloro-1-hydroxy-1H-indol-2-carboxylic acid (Example 3);
6-bromo-1-hydroxy-1H-indol-2-carboxylic acid (Example 4);
1-hydroxy-4-methyl-1H-indol-2-carboxylic acid (Example 5);
6-(3-carboxyphenyl)-1-hydroxy-1H-indol-2-carboxylic acid (Example 6);
1-hydroxy-6-[4-(methylsulfonyl)phenyl]-1H-indol-2-carboxylic acid (Example 7);
5-carbamoyl-1-hydroxy-1H-indol-2-carboxylic acid (Example 8);
5-fluoro-1-hydroxy-1H-indol-2-carboxylic acid (Example 9);
1-hydroxy-3-methyl-1H-indol-2-carboxylic acid (Example 10);
3-ethyl-1-hydroxy-1H-indol-2-carboxylic acid (Example 11);
5-(4-carboxy-1H-1,2,3-triazol-1-yl)-1-hydroxy-1H-indol-2-carboxylic acid (Example 12)
6-(4-carboxy-1H-1,2,3-triazol-1-yl)-1-hydroxy-1H-indol-2-carboxylic acid (Example 13);
6-[4-(2-carboxyethyl)-1H-1,2,3-triazol-1-yl]-1-hydroxy-1H-indol-2-carboxylic acid (Example 14);
6,6′-(4,4′-(propane-1,3-diyl)bis(1H-1,2,3-triazole-4,1-diyl))bis(1-hydroxy-1H-indole-2-carboxylic acid) (Example 15);
6-[4-(3-carboxypropyl)-1H-1,2,3-triazol-1-yl]-1-hydroxy-1H-indol-2-carboxylic acid (Example 16);
6-(4-carboxyphenyl)-1-hydroxy-1H-indol-2-carboxylic acid (Example 17);
6-[5-(3-carboxypropyl)-1H-1,2,3-triazol-1-yl]-1-hydroxy-1H-indol-2-carboxylic acid (Example 18);
1-hydroxy-5-[N-methyl-N-phenylcarbamoyl]-1H-indol-2-carboxylic acid (Example 19);
1-hydroxy-6-phenyl-4-trifluoromethyl-1H-indol-2-carboxylic acid (Example 20);
1-hydroxy-5-(morpholin-4-carbonyl)-1H-indol-2-carboxylic acid (Example 21);
1-hydroxy-4,4-(2-hydroxyethyl)-1H-1,2,3-triazol-1-yl]-1H-indol-2-carboxylic acid (Example 22);
1-hydroxy-5-(4-phenyl-1H-1,2,3-triazol-1-yl)-1H-indol-2-carboxylic acid (Example 23);
1-hydroxy-4-(4-phenyl-1H-1,2,3-triazol-1-yl)-1H-indol-2-carboxylic acid (Example 24);
1-hydroxy-6-[N-methyl-N-phenylcarbamoyl]-1H-indol-2-carboxylic acid (Example 25);
1-hydroxy-6-[N-methyl-N-phenylsulfamoyl]-1H-indol-2-carboxylic acid (Example 26);
6-(N,N-dimethylcarbamoyl)-1-hydroxy-1H-indol-2-carboxylic acid (Example 27);
6-(N,N-dimethylsulfamoyl)-1-hydroxy-1H-indol-2-carboxylic acid (Example 28);
6-carbamoyl-1-hydroxy-1H-indol-2-carboxylic acid (Example 29);
1-hydroxy-5-phenyl-1H-indol-2-carboxylic acid (Example 30);
1-hydroxy-6-(4-methoxyphenyl)-1H-indol-2-carboxylic acid (Example 31);
1-hydroxy-6-phenyl-1H-indol-2-carboxylic acid (Example 32);
1-hydroxy-1H-indol-2,5-dicarboxylic acid (Example 33);
6-fluoro-1-hydroxy-1H-indol-2-carboxylic acid (Example 34);
5-cyano-1-hydroxy-1H-indol-2-carboxylic acid (Example 35);
6-cyano-1-hydroxy-1H-indol-2-carboxylic acid (Example 36);
4-fluoro-1-hydroxy-1H-indol-2-carboxylic acid (Example 37);
1-hydroxy-4-trifluoromethyl-1H-indol-2-carboxylic acid (Example 38);
5-fluoro-1-hydroxy-6-phenyl-1H-indol-2-carboxylic acid (Example 39);
1-hydroxy-4-phenyl-1H-indol-2-carboxylic acid (Example 40);
4-(4-butyl-1H-1,2,3-triazol-1-yl)-1-hydroxy-1H-indol-2-carboxylic acid (Example 41);
1-hydroxy-6-[4-(2-hydroxyethyl)-1H-1,2,3-triazol-1-yl]-1H-indol-2-carboxylic acid (Example 42);
1-hydroxy-5-[4-(2-hydroxyethyl)-1H-1,2,3-triazol-1-yl]-1H-indol-2-carboxylic acid (Example 43);
5-(cyclopropylsulfonylcarbamoyl)-1-hydroxy-1H-indol-2-carboxylic acid (Example 44);
6-(cyclopropylsulfonylcarbamoyl)-1-hydroxy-1H-indol-2-carboxylic acid (Example 45);
1-hydroxy-6-(2H-tetrazol-5-yl)-1H-indol-2-carboxylic acid (Example 46);
5-[4-(2-carboxyethyl)phenyl]-1-hydroxy-1H-indol-2-carboxylic acid (Example 47);
4-[4-(3-carboxyphenyl)-1H-1,2,3-triazol-1-yl]-1-hydroxy-1H-indol-2-carboxylic acid (Example 48);
6-[4-(2-carboxyethyl)phenyl]-1-hydroxy-1H-indol-2-carboxylic acid (Example 49);
6-[4-(4-carboxyphenyl)-1H-1,2,3-triazol-1-yl]-1-hydroxy-1H-indol-2-carboxylic acid (Example 50);
5-(4-chlorophenoxy)-1-hydroxy-1H-indol-2-carboxylic acid (Example 51);
5-(4-butyl-1H-1,2,3-triazol-1-yl)-1-hydroxy-1H-indol-2-carboxylic acid (Example 52);
1-hydroxy-6-[4-(pyridin-2-yl)-1H-1,2,3-triazol-1-yl]-1H-indol-2-carboxylic acid (Example 53);
6-[4-(carboxycarbonylcarbamoyl)phenyl]-1-hydroxy-1H-indol-2-carboxylic acid (Example 54);
1-hydroxy-6-(5-oxo-4,5-dihydro-1,2,4-oxadiazol-3-yl)-1H-indol-2-carboxylic acid (Example 55);
5-(3-carboxyphenyl)-1-hydroxy-1H-indol-2-carboxylic acid (Example 56);
1-hydroxy-5,6-diphenyl-1H-indole-2-carboxylic acid (Example 57);
1-hydroxy-6-(N-methyl-N-p-tolylsulfamoyl)-1H-indole-2-carboxylic acid (Example 58);
1-hydroxy-6-(N-methyl-N-(4-(trifluoromethyl)phenyl)sulfamoyl)-1H-indole-2-carboxylic acid (Example 59);
6-(N-(4-fluorophenyl)-N-methylsulfamoyl)-1-hydroxy-1H-indole-2-carboxylic acid (Example 60);
6-(N-(4-chlorophenyl)-N-methylsulfamoyl)-1-hydroxy-1H-indole-2-carboxylic acid (Example 61);
5-(4-(3-carboxyphenyl)-1H-1,2,3-triazol-1-yl)-1-hydroxy-1H-indole-2-carboxylic acid (Example 62);
1-hydroxy-6-(4-(trifluoromethyl)phenyl)-1H-indole-2-carboxylic acid (Example 63);
6-(4-fluorophenyl)-1-hydroxy-1H-indole-2-carboxylic acid (Example 64);
5-(4-fluorophenyl)-1-hydroxy-1H-indole-2-carboxylic acid (Example 65);
1-hydroxy-5-(4-(trifluoromethyl)phenyl)-1H-indole-2-carboxylic acid (Example 66);
6-(benzo[d][1,3]dioxol-5-yl)-1-hydroxy-1H-indole-2-carboxylic acid (Example 67);
1-hydroxy-5-(4-methoxyphenyl)-1H-indole-2-carboxylic acid (Example 68);
6-(N-(2-chlorophenyl)-N-methylsulfamoyl)-1-hydroxy-1H-indole-2-carboxylic acid (Example 69);
6-(2,2-difluorobenzo[d][1,3]dioxol-5-yl)-1-hydroxy-1H-indole-2-carboxylic acid (Example 70);
5-(4-chlorophenyl)-1-hydroxy-1H-indole-2-carboxylic acid (Example 71);
6-(4-chlorophenyl)-1-hydroxy-1H-indole-2-carboxylic acid (Example 72);
1-hydroxy-6,7-diphenyl-4-(trifluoromethyl)-1H-indole-2-carboxylic acid (Example 73)
6-(N-butyl-N-phenylsulfamoyl)-1-hydroxy-1H-indole-2-carboxylic acid (Example 74);
6-(4-(N,N-dimethylsulfamoyl)phenyl)-1-hydroxy-1H-indole-2-carboxylic acid (Example 75);
6-(furan-3-yl)-1-hydroxy-1H-indole-2-carboxylic acid (Example 76);
1-hydroxy-6-(3-(trifluoromethoxy)phenyl)-1H-indole-2-carboxylic acid (Example 77);
6-(4-chlorophenyl)-1-hydroxy-4-(trifluoromethyl)-1H-indole-2-carboxylic acid (Example 78);
6-(biphenyl-4-yl)-1-hydroxy-1H-indole-2-carboxylic acid (Example 79);
1-hydroxy-3-methyl-6-phenyl-4-(trifluoromethyl)-1H-indole-2-carboxylic acid (Example 80);
1-hydroxy-6-(4-(trifluoromethoxy)phenyl)-1H-indole-2-carboxylic acid (Example 81);
1-hydroxy-6-(4-(N-methyl-N-phenylsulfamoyl)phenyl)-1H-indole-2-carboxylic acid (Example 82);
6-(4-chlorophenyl)-1-hydroxy-3-methyl-4-(trifluoromethyl)-1H-indole-2-carboxylic acid (Example 83);
1-hydroxy-6-(naphthalen-1-yl)-1H-indole-2-carboxylic acid (Example 84);
1-hydroxy-6-(naphthalen-2-yl)-1H-indole-2-carboxylic acid (Example 85);
6-(2,4-dichlorophenyl)-1-hydroxy-4-(trifluoromethyl)-1H-indole-2-carboxylic acid (Example 86);
6-(N-(3-chlorophenyl)-N-methylsulfamoyl)-1-hydroxy-1H-indole-2-carboxylic acid (Example 87);
1-hydroxy-5-(N-methyl-N-phenylsulfamoyl)-1H-indole-2-carboxylic acid (Example 88);
1-hydroxy-1H-benzo[d]imidazole-2-carboxylic acid (Example 89);
2-carboxy-3-hydroxy-3H-benzo[d]imidazole 1-oxide (Example 90);
2-carboxy-5-chloro-3-hydroxy-3H-benzo[d]imidazole 1-oxide (Example 91);
2-carboxy-3-hydroxy-5-phenyl-3H-benzo[d]imidazole 1-oxide (Example 92);
2-(2-(benzoylimino)-3-hydroxy-2,3-dihydrothiazol-4-yl)acetic acid (Example 93);
2-(2-(acetylimino)-3-hydroxy-2,3-dihydrothiazol-4-yl)acetic acid (Example 94);
4-(4-(carboxymethyl)-3-hydroxythiazol-2(3H)-ylidenecarbamoyl)benzoic acid (Example 95);
3-(4-(carboxymethyl)-3-hydroxythiazol-2(3H)-ylidenecarbamoyl)benzoic acid (Example 96);

### Compounds Synthesis

Examples 1-96 above, each of which constitutes an embodiment of this invention, may be prepared following the procedures reported below, which the skilled man in the art of organic chemistry may modify in order to obtain the same compounds without exercising any inventive skills.

The temperature below reported are always expressed as Celsius degrees.

The following abbreviations, reagents, expressions or equipments, which are utilized in the following description, are explained as follows: 20-25° C. (room temperature, RT), molar equivalents (eq.), N,N-dimethylformamide (DMF), 1,2-dimetoxyethane (DME), dichloromethane (DCM), chloroform (CHCl₃), ethylacetate (EtOAc), tetrahydrofuran (THF), methanol (MeOH), diethylether (Et₂O), dimethylsulfoxide (DMSO), sodium hydride (NaH), dimethyl oxalate (“(COOMe)₂”), stannous chloride dihydrate (SnCl₂. 2H₂O), sodium hypophosphite monohydrate (H₂PO₂Na.H₂O), palladium 10% on charcoal (Pd—C), lithium hydroxide (LiON), hydrochloric acid (HCl), acetic acid (AcOH), diethylamine (Et₂NH), triethylamine (Et₃N), sodium bicarbonate (NaHCO₃), normal concentration (N), millimoles (mmol), aqueous solution (aq.), thin layer chromatography (TLC), nuclear magnetic resonance (NMR), electronic impact mass spectrometry (EI/MS).

Examples 1-88 were prepared as shown in the general pathway of Scheme 1 and as reported in the following described methodologies.

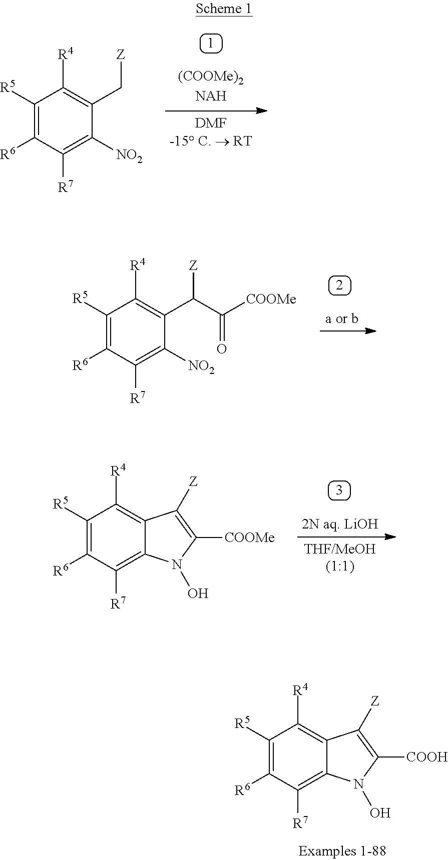

where:
a: SnCl₂.2H₂O, molecular sieves 4 Å, DME, RT;

### b: H₂PO₂Na.H₂O, Pd—C, H₂O/THF (1:1), RT

Step 1.

A suspension of sodium hydride (6 mmol) in 5 mL of anhydrous DMF cooled to −15° C. under nitrogen is treated dropwise with a solution containing the appropriate orto-alkyl-nitroaryl precursor (1.5 mmol) and dimethyl oxalate (7.5 mmol) in 4 mL of anhydrous DMF. The mixture is left under stirring at the same temperature for 10 minutes and then is slowly warmed to room temperature. After a certain time, which depends on the substrate, it is possible to observe the development of an intense colour, varying from cherry red to violet-blue. The mixture is then left under stirring at room temperature for 2-18 hours. Once the disappearance of the precursor is verified by TLC, the reaction mixture is slowly poured in an ice-water mixture; the water phase is acidified with 1N HCl and extracted several times with EtOAc. The combined organic phase is washed with 6% aqueous NaHCO₃, brine, and dried over anhydrous sodium sulphate. Evaporation under vacuum of the organic solvent affords a crude product which is purified by column chromatography over silica gel using n-hexane/EtOAc mixtures as the eluent, to yield the nitroaryl-ketoester derivative, which is utilized in the following step.

Step 2 Conditions a.

Classical methodologies describing the reductive cyclization of the nitroaryl-ketoester intermediate, which utilize SnCl₂.2H₂O, [Dong W, Jimenez L S, J. Org. Chem. 1999, 64, 2520-2523], were followed for the preparation of some reported examples 1-88. Briefly, a solution of nitroaryl-ketoester precursor deriving from step 1 in anhydrous DMF, in the presence of 4 Å molecular sieves previously activated for 18 hours at 130° C. in oven and cooled to RT in a dessiccator over either anhydrous calcium chloride or phosphoric anhydride, was treated with 2.2 eq. of SnCl₂.2H₂O at room temperature. The resulting suspension was kept under stirring in the dark for 2-24 hours. Once the disappearance of the precursor is verified by TLC, the reaction mixture is diluted with water and extracted several times with EtOAc. The combined organic phase is washed with brine and dried over anhydrous sodium sulphate. Evaporation under vacuum of the organic solvent affords a crude product which is purified by column chromatography over silica gel using n-hexane/EtOAc mixtures as the eluent, to yield the N-hydroxyindol-ester derivative, which is utilized in the following step.

Step 2—Conditions b.

The above reported conditions (conditions a) in some examples afforded large amounts (even higher than 90%) of side products due to over-reduction of the nitro group (NH-indol-ester derivatives), which lowered the yields of this step and were often very difficult to separate from the desired N—OH-indole product. Therefore, we searched for another synthetic methodology, in order to dramatically reduce the occurrence of this side reaction. We, then, replaced the previously used reducing agent (SnCl₂.2H₂O) with a combination of H₂PO₂Na.H₂O and Pd—C. This reducing system had already been successfully utilized in the past for the selective reduction of nitro-groups to hydroxylamines [Entwistle I D, et. al. Tetrahedron 1978, 34, 213-215], but it was not used for the preparation of N-hydroxyindole systems like ours. In details, an aqueous solution (0.6 mL) containing 1.1 mmol of H₂PO₂Na.H₂O is treated at RT with another solution containing the nitroaryl-ketone precursor (0.35 mmol) in THF; 3.5 mg of Pd—C are added to the resulting mixture and the mixture is kept under stirring at the same temperature for 12-20 hours. Once the disappearance of the precursor is verified by TLC, the reaction mixture is diluted with water and extracted several times with EtOAc. The combined organic phase is washed with brine and dried over anhydrous sodium sulphate. Evaporation under vacuum of the organic solvent affords a crude product which is purified by column chromatography over silica gel using n-hexane/EtOAc mixtures as the eluent, to yield the N-hydroxyindol-ester derivative, which is utilized in the following step.

Below are reported three cases where conditions b proved to be effective in reducing the amounts of the over-reduced side products, when compared to conditions a (FIG. 1).

### FIG. 1.

Synthesis of Example 15

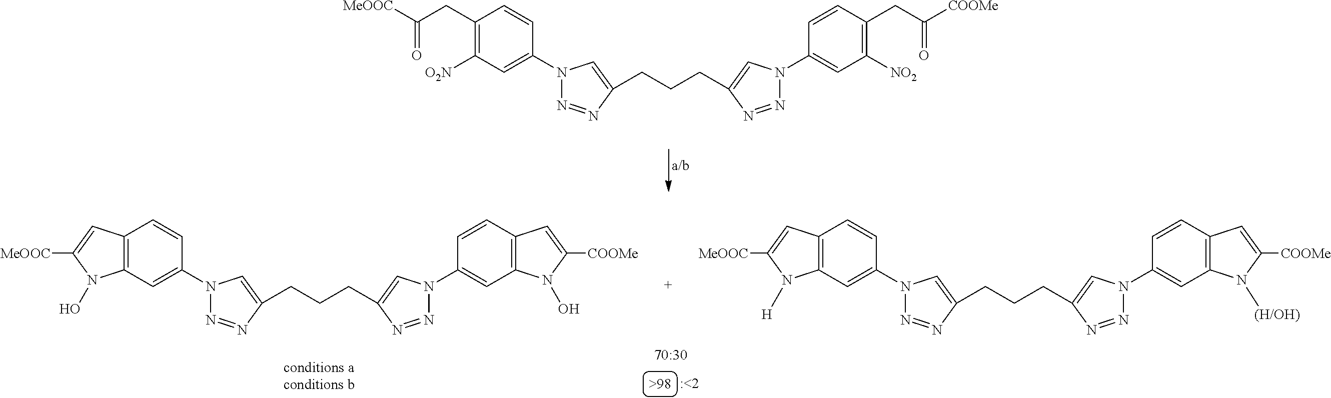

Synthesis of Example 26

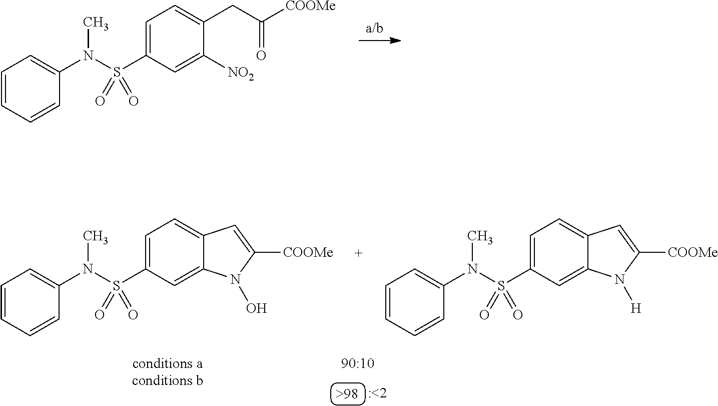

Synthesis of Example 47

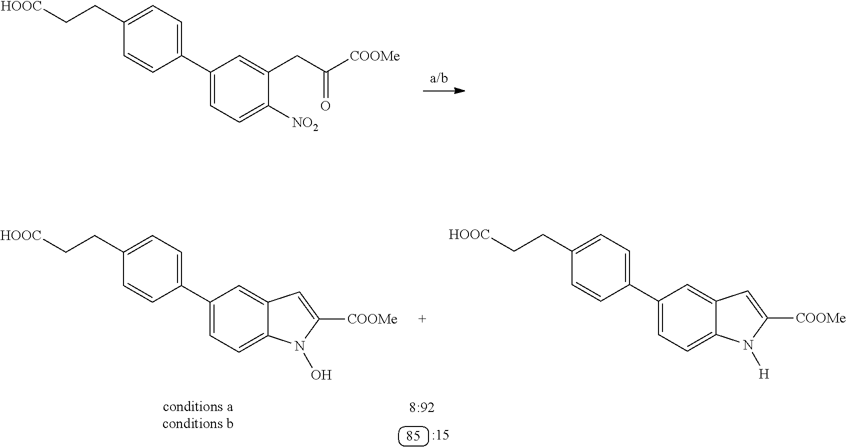

Step 3.

A solution of the N-hydroxyindol-ester intermediate (0.25 mmol) in 2.5 mL of a 1:1 mixture of MeOH and THF is treated at RT with 0.8 mL of an aqueous 2N solution of LiOH. The reaction mixture is left under stirring in the dark at the same temperature for 12-24 hours. Once the disappearance of the precursor is verified by TLC, the reaction mixture is diluted with water, acidified with a aqueous 1N solution of HCl, and extracted several times with EtOAc. The combined organic phase is washed with brine and dried over anhydrous sodium sulphate. Evaporation under vacuum of the organic solvent affords the final N-hydroxyindol-carboxylic acid product (Example 1-88).

Example 89 had been previously reported for purpose that are completely different from those claimed in the present invention [Seng F, Ley K. Synthesis 1975, 11, 703]. We have now synthesized it following a procedure (Scheme 2) previously reported for other analogues of Example 89 [McFarlane M D, Moody D J, Smith D M. J. Chem. Soc. Perkin Trans. I 1988, 691-696].

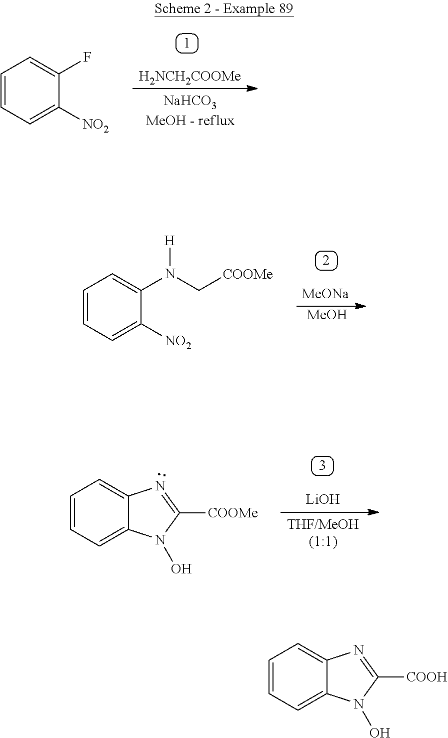

Step 1.

A solution containing methyl glycinate hydrochloride (7.1 mmol), 1-fluoro-2-nitrobenzene (7.1 mmol) and sodium bicarbonate (14.2 mmol) in methanol (8 mL) is heated to reflux for 24 hours. Evaporation under vacuum of methanol affords a crude product which is taken up with H₂O and EtOAc. The organic phase is washed with brine and dried over anhydrous sodium sulphate. Evaporation under vacuum of the organic solvent affords a crude product which is purified by column chromatography over silica gel using a 9:1 n-hexane/EtOAc mixture as the eluent, to yield the N-arylglycinic derivative, which is then utilized in the following step.

Step 2.

A freshly prepared solution of sodium methoxide (0.90 mmol) in MeOH (5 mL) is treated with the N-arylglycinic derivative (0.33 mmol) prepared in the previous step. The resulting mixture is left under stirring at RT for 5 hours. Once the disappearance of the glycinic precursor is verified by TLC, the reaction mixture is diluted with water and acidified with AcOH. The resulting suspension is extracted several times with Et₂O. The combined organic phase is washed with brine and dried over anhydrous sodium sulphate. Evaporation under vacuum of the organic solvent affords a crude product which is purified by column chromatography over silica gel using a 3:7 mixture of n-hexane/EtOAc as the eluent, to yield the N-hydroxybenzimidazol-ester derivative, which is utilized in the following step.

Step 3.

A solution containing the N-hydroxybenzimidazol-ester derivative (0.41 mmol) in 4 mL of a 1:1 mixture of MeOH and THF is treated at RT with 1.2 of an aqueous 2N solution of LiOH. The reaction mixture is left under stirring in the dark at the same temperature for 2 hours. Once the disappearance of the precursor is verified by TLC, most of the organic solvent is evaporated under vacuum and the reaction mixture is diluted with water, acidified with a aqueous 1N solution of HCl, and extracted several times with EtOAc. The combined organic phase is washed with brine and dried over anhydrous sodium sulphate. Evaporation under vacuum of the organic solvent affords the final N-hydroxybenzimidazol-carboxylic acid product (Example 89).

Example 90, previously reported for purpose that are completely different from those claimed in the present invention, was synthesized as described in the art [Claypool D P, Sidani A R, Flanagan K J. J. Org. Chem. 1972, 37, 2372-2376], whereas its analogues, Examples 91 and 92, are new molecules, which were instead synthesized by following a procedure previously developed for similar compounds [El-Haj M J A. J. Org. Chem. 1972, 37, 2519-2520], whose synthesis is shown in Scheme 3.

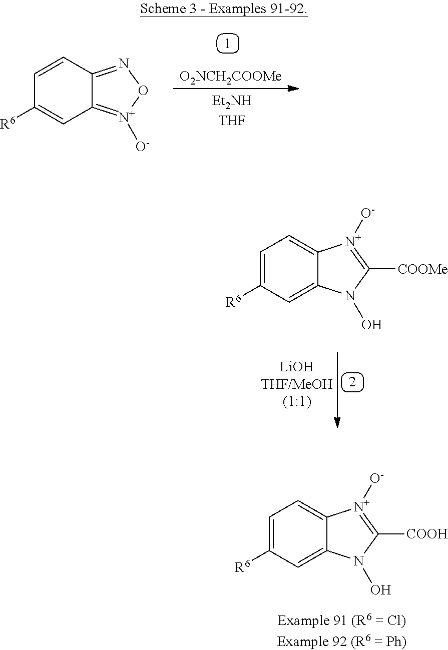

Step 1.

A solution containing the properly substituted benzofurazan-oxide precursor (2.1 mmol) and methyl nitroacetate (2.5 mmol) in THF (2 mL) was slowly treated at RT with Et₂NH (2.5 mmol). After completion of the addition, the resulting mixture was left under stirring for 24 hours. Then, the reaction mixture is diluted with water, acidified with a aqueous 1N solution of HCl, and to extracted several times with EtOAc. The combined organic phase is washed with brine and dried over anhydrous sodium sulphate. Evaporation under vacuum of the organic solvent affords a crude product which is purified by column chromatography over silica gel using n-hexane/EtOAc mixtures as the eluent, to yield the N-hydroxyindol-N-oxide-ester derivative, which is utilized in the following step.

Step 2.

A solution of the N-hydroxyindol-N-oxide-ester intermediate (0.40 mmol) in 3 mL of a 1:1 mixture of MeOH and THF is treated at RT with 1.2 of an aqueous 2N solution of LiOH. The reaction mixture is left under stirring in the dark at the same temperature for 2 hours. Once the disappearance of the precursor is verified by TLC, most of the organic solvent is evaporated under vacuum and the reaction mixture is diluted with water, acidified with a aqueous 1N solution of HCl, and extracted several times with EtOAc. The combined organic phase is washed with brine and dried over anhydrous sodium sulphate. Evaporation under vacuum of the organic solvent affords the final N-hydroxybenzimidazol-N-oxide-carboxylic acid product (Examples 91-92).

Examples 93-96 are all constituted by novel compounds and their synthesis is shown in Scheme 4.

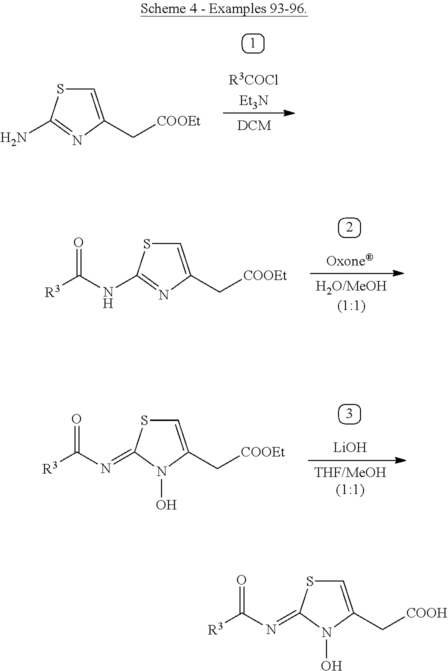

Step 1.

A DCM solution of commercially available ethyl 2-(2-aminothiazol-4-yl)acetate (5.4 mmol), cooled to 0° C., is treated with the appropriate acyl chloride (11 mmol) and triethylamine (6.4 mmol). The reaction mixture is then warmed to RT and kept under stirring for 16-18 hours. Once the disappearance of the amine precursor is verified by TLC, the mixture is washed with H₂O and a saturated aqueous solution of NaHCO₃, then dried over anhydrous sodium sulphate and concentrated under vacuum. The resulting crude product is purified by column chromatography over silica gel using n-hexane/EtOAc mixtures as the eluent, to yield the N-acylated derivative, which is utilized in the following step.

Step 2.

The N-acylated thiazol derivative (2.2 mmol) is dissolved in 40 mL of a 1:1 mixture of H₂O and MeOH; the resulting solution is cooled to 0° C. and treated with Oxone® (4.6 mmol), an oxidative reagent which is commercially available under that registered name. The reaction mixture is left under stirring in the dark at RT for 24 hours and, after that, most of the THF is removed by evaporation under vacuum. The resulting crude residue is diluted with H₂O, and extracted several times with EtOAc. The combined organic phase is washed with brine, dried over anhydrous sodium sulphate and concentrated under vacuum. The resulting crude product is purified by column chromatography over silica gel using CHCl₃/MeOH mixtures as the eluent, to yield the N-hydroxylated ester derivative, which is utilized in the following step.

Step 3.

A solution of the N-hydroxythiazol-ester intermediate (0.52 mmol) in 5 mL of a 1:1 mixture of MeOH and THF is treated at RT with 1.6 of an aqueous 2N solution of LiOH. The reaction mixture is left under stirring in the dark at the same temperature for 16-24 hours. Once the disappearance of the ester precursor is verified by TLC, most of the organic solvent is evaporated under vacuum and the reaction mixture is diluted with water, acidified with a aqueous 1N solution of HCl, and extracted several times with EtOAc. The combined organic phase is washed with brine and dried over anhydrous sodium sulphate. Evaporation under vacuum of the organic solvent affords the final N-hydroxythiazol-carboxylic acid product (Examples 93-96).

### Characterization Data

Below are reported the characterization data of compounds indicated in Examples 1-96
NMR spectra were obtained with a Varian Gemini 200 MHz spectrometer. Chemical shifts (δ) are reported in parts per million downfield from tetramethylsilane and referenced from solvent references. Electron impact (EI, 70 eV) mass spectra were obtained on a Thermo Quest Finningan (TRACE GCQ plus MARCA) mass spectrometer. Purity was routinely measured by HPLC on a Waters SunFire RP 18 (3.0×150 mm, 5 μm) column (Waters, Milford, Mass., www.waters.com) using a Beckmann SystemGold instrument consisting of chromatography 125 Solvent Module and a 166 UV Detector. Mobile phases: 10 mM ammonium acetate in Millipore purified water (A) and HPLC grade acetonitrile (B). A gradient was formed from 5% to 80% of B in 10 minutes and held at 80% for 10 min; flow rate was 0.7 mL/min and injection volume was 30 μL; in some examples, retention times (HPLC, t_{R}) are given in minutes.

### Example 1

¹H NMR (DMSO-d₆, 200 MHz): δ 7.00 (d, 1H, J=0.9 Hz), 7.08 (ddd, 1H, J=8.1, 6.8, 1.1 Hz), 7.31 (ddd, 1H, J=8.4, 6.8, 1.1 Hz), 7.43 (dq, 1H, J=8.4, 1.1 Hz), 7.63 (dt, 1H, J=8.1, 1.0 Hz), 11.73 (bs, 1H). ¹³C NMR (DMSO-d₆) δ 106.17, 110.38, 121.52, 122.96, 123.14, 126.03, 126.38, 136.92, 162.25. MS m/z 177 (M⁺, 100%), 161 159 (M⁺-O, 28%), 159 (M⁺-H₂O, 13%), 133 (M⁺-CO₂, 5%), 115 (M⁺-H₂O—CO₂, 44%). HPLC, t_{R} 7.1 min.

### Example 2

¹H NMR (DMSO-d₆, 200 MHz): δ 6.88 (d, 1H, J=0.9 Hz), 7.23 (t, 1H, J=7.8 Hz), 7.35 (dd, 1H, J=7.4, 1.0 Hz), 7.48 (dt, 1H, J=8.1, 1.0 Hz). ¹³C NMR (acetone-d₆) δ 105.06, 110.13, 116.53, 122.72, 124.29, 126.82, 127.38, 136.87, 161.61. MS m/z 257 (⁸¹Br: M⁺, 91%), 255 (⁷⁹Br: M⁺, 100%), 241 (⁸¹Br: M⁺-O, 5%), 239 (⁷⁹Br: M⁺-O, 8%), 114 (M⁺-H₂O—CO₂—Br, 66%). HPLC, t_{R} 8.5 min.

### Example 3

¹H NMR (DMSO-d₆, 200 MHz): δ 6.97 (s, 1H), 7.19 (dd, 1H, J=7.3, 0.9 Hz), 7.31 (t, 1H, J=7.8 Hz), 7.44 (d, 1H, J=8.2 Hz). ¹³C NMR (acetone-d₆) δ 103.64, 109.57, 121.14, 123.80, 126.67, 127.22, 127.82, 137.29, 161.65. MS m/z 211 (M⁺, 100%), 195 (M⁺-O, 10%), 149 (M⁺-H₂O—CO₂, 13%), 114 (M⁺-H₂O—CO₂—Cl, 34%). HPLC, t_{R} 7.9 min.

### Example 4

¹H NMR (DMSO-d₆, 200 MHz): δ 7.03 (d, 1H, J=0.7 Hz), 7.22 (dd, 1H, J=8.0, 1.7 Hz), 7.59-7.63 (m, 2H). ¹³C NMR (acetone-d₆) δ 106.35, 113.07, 119.37, 121.32, 124.83, 124.92, 127.42, 137.38, 161.67. MS m/z 257 (⁸¹Br: M⁺, 93%), 255 (⁷⁹Br: M⁺, 100%), 241 (⁸¹Br: M⁺-O, 4%), 239 (⁷⁹Br: M⁺-O, 7%), 114 (M⁺-H₂O—CO₂—Br, 39%). HPLC, t_{R} 8.3 min.

### Example 5

¹H NMR (DMSO-d₆) δ 2.48 (s, 3H), 6.88 (d, 1H, J=6.4 Hz), 7.02 (s, 1H), 7.15-7.27 (m, 2H), 11.37 (bs, 1H). ¹³C NMR (CD₃OD) δ 18.14, 105.06, 108.14, 121.51, 122.83, 126.37, 126.55, 132.72, 137.66, 163.86. MS m/z 191 (M⁺, 100%), 175 (M⁺-O, 6%), 146 (M⁺-CO₂—H, 5%), 129 (M⁺-H₂O—CO₂, 19%). HPLC, t_{R} 7.9 min.

### Example 6

¹H NMR (DMSO-d₆): δ 7.06 (d, 1H, J=0.7 Hz), 7.46 (dd, 1H, J=8.4, 1.0 Hz), 7.62 (t, 1H, J=7.7 Hz), 7.76 (d, 1H, J=8.1 Hz), 7.92-8.02 (m, 3H), 8.24 (t, 1H, J=3.0 Hz).

### Example 7

¹H NMR (DMSO-d₆): δ 3.26 (s, 3H), 7.06 (s, 1H), 7.50 (dd, 1H, J=8.2, 1.4 Hz), 7.76-7.80 (m, 2H), 8.01 (s, 1H). ¹³C NMR (DMSO-d₆): δ 43.65, 104.40, 107.95, 119.89, 121.15, 122.95, 127.65 (2C), 127.78 (2C), 131.42, 134.89, 136.17, 139.21, 145.49, 161.06.

### Example 8

¹H NMR (DMSO-d₆, 200 MHz): δ 7.11 (d, 1H, J=0.5 Hz), 7.23 (bs, 1H), 7.45 (d, 1H, J=8.6 Hz), 7.84 (dd, 1H, J=8.8, 1.5 Hz), 7.93 (bs, 1H), 8.24 (s, 1H).

### Example 9

¹H NMR (DMSO-d₆): δ 6.98 (s, 1H), 7.18 (td, 1H, J=9.2, 2.4), 7.39-7.48 (m, 2H). EI/MS (70 eV) m/z 195 (M⁺, 100%), 133 (M⁺-CO₂—H₂O, 28%).

### Example 10

¹H NMR (DMSO-d₆): δ 2.48 (s, 3H), 7.01 (td, 1H, J=7.3, 1.5 Hz), 7.30 (td, 1H, J=7.4, 1.1 Hz), 7.35-7.40 (m, 1H), 7.64 (d, 1H, J=7.9 Hz), 11.03 (bs, 1H).

### Example 11

¹H NMR (DMSO-d₆): δ 1.17 (t, 3H, J=7.3 Hz), 2.99 (q, 2H, J=7.4 Hz), 7.07 (td, 1H, J=7.3, 0.9 Hz), 7.26-7.40 (m, 2H), 7.66 (d, 1H, J=7.9 Hz), 12.00 (bs, 1H).

### Example 12

¹H NMR (DMSO-d₆): δ 7.14 (s, 1H), 7.65 (d, 1H, J=9.0 Hz), 7.87 (dd, 1H, J=9.0, 2.2 Hz), 8.23 (d, 1H, J=2.0 Hz), 9.32 (s, 1H), 12.13 (bs, 1H).

### Example 13

¹H NMR (DMSO-d₆): δ 7.13 (s, 1H), 7.71 (dd, 1H, J=8.6, 1.8 Hz), 7.87 (d, 1H, J=8.6 Hz), 8.04 (d, 1H, J=1.4 Hz), 9.48 (s, 1H).

### Example 14

¹H NMR (DMSO-d₆): δ 2.69 (t, 2H, J=7.4 Hz), 2.95 (t, 2H, J=7.3 Hz), 7.10 (s, 1H), 7.64 (dd, 1H, J=8.5, 1.9 Hz), 7.52 (d, 1H, J=9.0 Hz), 7.9 (d, 1H, J=2.3 Hz), 8.70 (s, 1H), 12.10 (bs, 1H). ¹³C NMR (DMSO-d₆): δ 20.98, 33.17, 98.22, 101.03, 115.86, 115.96 (2C), 120.95, 124.24, 134.26, 135.84, 149.81, 161.08, 173.93.

### Example 15

¹H NMR (DMSO-d₆): δ 2.13 (t, 2H, J=7.4. Hz), 2.85 (t, 4H, J=7.1 Hz), 7.11 (s, 2H), 7.67 (dd, 2H, J=8.8, 1.6 Hz), 7.84 (d, 2H, J=8.6 Hz), 7.93 (d, 2H), 8.77 (s, 2H). ¹³C NMR (DMSO-d₆): δ 24.64 (2C), 28.45 (1C), 100.52 (2C), 104.87 (2C), 113.17 (2C), 120.49 (4C), 123.68 (2C), 128.39 (2C), 134.00 (2C), 135.53 (2C), 147.64 (2C), 160.80 (2C). MS m/z 546 (M+NH₄⁺, 5%), 256 ((M+NH₄⁺)/2-OH, 40%), 256 ((M+NH₄⁺)/2-2OH, 100%).

### Example 16

¹H NMR (DMSO-d₆): δ 1.84-1.99 (m, 2H), 2.30-2.38 (m, 2H), 2.70-2.78 (m, 2H), 7.10 (d, 1H, J=0.9 Hz), 7.67 (dd, 1H, J=8.6, 1.8 Hz), 7.83 (d, 1H, J=8.6 Hz), 7.91-7.93 (m, 1H), 8.74 (s, 1H), 12.12 (bs, 1H).

### Example 17

¹H NMR (acetone-d₆): δ 7.04 (s, 1H), 7.49 (dd, 1H, J=8.4, 1.4 Hz), 7.76 (d, 1H, J=8.6 Hz), 7.83 (d, 1H, J=1.2 Hz), 7.88-7.92 (m, 2H), 8.13-8.17 (m, 2H).

### Example 18

¹H NMR (acetone-d₆): δ 1.34-1.38 (m, 2H), 2.41-2.48 (m, 2H), 2.82-2.89 (m, 2H), 7.20 (d, 1H, J=0.9 Hz), 7.70 (dd, 1H, J=8.6, 2.0 Hz), 7.87 (d, 1H, J=8.6 Hz), 8.02-8.03 (m, 1H), 8.48 (s, 1H), 11.24 (bs, 1H).

### Example 19

¹H NMR (DMSO-d₆, 200 MHz): δ 1.91 (s, 3H), 6.94 (s, 1H), 7.10-7.27 (m, 6H), 7.60-7.64 (m, 2H), 11.85 (bs, 1H).

### Example 20

¹H NMR (acetone-d₆): δ 7.20 (qd, 1H, J=1.8, 0.7 Hz), 7.43-7.58 (m, 3H), 7.80-7.85 (m, 3H), 8.04-8.06 (m, 1H). ¹³C NMR (acetone-d₆): δ 103.43, 112.32, 117.22, 119.01 (q, J=4.8 Hz), 123.81 (q, J=33.0 Hz), 125.47 (q, J=262.8 Hz), 128.07 (2C), 128.71, 129.89 (2C), 133.64, 137.54, 138.52, 140.71, 161.45. MS m/z 321 (M⁺, 100%), 305 (M⁺-O, 10%), 259 (M⁺-H₂O—CO₂, 41%), 190 (M⁺-H₂O—CO₂—CF₃, 38%). HPLC, t_{R}=10.5 min.

### Example 21

¹H NMR (DMSO-d₆): δ 3.53-3.59 (m, 8H), 7.08 (d, 1H, J=0.7 Hz), 7.35 (dd, 1H, J=8.6, 1.5 Hz), 7.47 (d, 1H, J=8.6 Hz), 7.74 (d, 1H, J=1.6 Hz).

### Example 22

¹H NMR (DMSO-d₆): δ 2.90 (t, 2H, J=7.0 Hz), 3.74 (t, 2H, J=6.9 Hz), 7.32 (d, 1H, J=0.7 Hz), 7.46-7.52 (m, 2H), 7.56-7.60 (m, 1H), 8.60 (s, 1H). ¹³C NMR (DMSO-d₆): δ 29.18, 60.22, 103.39, 110.13, 112.54, 113.34, 121.95, 124.88, 127.70, 129.94, 137.04, 145.05, 160.70. MS m/z 287 (M⁺-H).

### Example 23

¹H NMR (DMSO-d₆): δ 7.16 (s, 1H), 7.34-7.54 (m, 3H), 7.67 (d, 1H, J=8.8 Hz), 7.88 (dd, 1H, J=8.8, 2.0 Hz), 7.95-7.99 (m, 2H), 8.20 (d, 1H, J=1.8 Hz), 9.29 (s, 1H). MS m/z 321 (M+H⁺, 10%), 287 (M⁺-O—OH, 100%).

### Example 24

¹H NMR (DMSO-d₆): δ 7.35-7.44 (m, 2H), 7.48-7.56 (m, 3H), 7.59-7.65 (m, 2H), 8.00-8.05 (m, 2H), 9.35 (s, 1H). ¹³C NMR (DMSO-d₆): δ 103.25, 110.51, 112.86, 113.41, 120.82, 124.84, 125.44 (2C), 127.88, 128.21, 128.92 (2C), 129.78, 130.20, 136.99, 146.77, 160.77. MS m/z 321 (M+H⁺).

### Example 25

¹H NMR (DMSO-d₆): δ 3.40 (s, 3H), 6.91 (d, 1H, J=0.7 Hz), 6.94 (dd, 1H, J=8.2, 1.5 Hz), 7.12-7.28 (m, 5H), 7.36-7.37 (m, 1H), 7.42 (d, 1H, J=8.4 Hz).

### Example 26

¹H NMR (DMSO-d₆): δ 3.15 (s, 3H); 7.08-7.15 (m, 4H); 7.27-7.34 (m, 3H); 7.56-7.57 (m, 1H), 7.79 (d, 1H, J=8.2 Hz). ¹³C NMR (DMSO-d₆): δ 30.69, 104.21, 109.97, 118.31, 122.89, 123.53, 126.12 (2C), 127.08, 128.78 (2C), 130.01, 131.67, 133.87, 141.14, 160.62. MS m/z 346 (M⁺, 17%), 330 (M⁺-O, 14%), 240 (M⁺-PhNMe, 10%), 224 (M⁺-O-PhNMe, 18%), 177 (M⁺-PhNMe-SO₂+H, 51%), 106 (PhNMe⁺, 100%).

### Example 27

¹H NMR (DMSO-d₆): δ 2.97 (s, 6H), 7.04-7.13 (m, 2H), 7.43-7.44 (m, 1H), 7.68 (d, 1H, J=8.2 Hz), 11.94 (s, 1H).

### Example 28

¹H NMR (DMSO-d₆): δ 2.62 (s, 6H), 7.15 (d, 1H, J=0.7 Hz), 7.41 (dd, 1H, J=8.4, 1.6 Hz), 7.77 (d, 1H, J=1.1 Hz), 7.89 (d, 1H, J=8.6 Hz). MS m/z 284 (M⁺, 34%), 282 (M⁺-H₂, 100%).

### Example 29

¹H NMR (DMSO-d₆): δ 7.02 (d, 1H, J=0.7 Hz), 7.32 (bs, 2H), 7.61 (dd, 1H, J=8.6, 1.5 Hz), 7.67 (d, 1H, J=8.4 Hz), 7.99 (s, 1H).

### Example 30

¹H NMR (DMSO-d₆): δ 7.04 (d, 1H, J=0.9 Hz), 7.32-7.36 (m, 1H), 7.42-7.53 (m, 2H), 7.60-7.60 (m, 4H), 7.90 (t, 1H, J=0.9 Hz). ¹³C NMR (DMSO-d₆): δ 104.51, 110.10, 119.85, 119.93, 121.46, 124.10, 126.66 (2C), 127.34, 128.85 (2C), 132.67, 135.00, 140.94, 161.39. MS m/z 253 (M⁺, 100%), 237 (M⁺-O, 40%), 190 (M⁺-H₂O—CO₂—H, 62%), 165 (M⁺-H₂O—CO₂—C₂H₂, 12%). HPLC, t_{R} 9.4 min.

### Example 31

¹H NMR (DMSO-d₆): δ 3.81 (s, 3H), 7.02-7.06 (m, 3H), 7.38 (dd, 1H, J=8.3, 1.6 Hz), 7.57 (d, 1H, J=1.4 Hz), 7.64-7.70 (m, 3H). ¹³C NMR (DMSO-d₆): δ 55.18, 104.61, 106.34, 114.36, 119.71, 119.91, 122.53, 127.03, 127.88, 132.93, 136.73, 141.67, 158.71, 161.03. MS m/z 283 (M⁺, 21%), 267 (M⁺-O, 100%).

### Example 32

¹H NMR (DMSO-d₆): δ 7.03 (s, 1H), 7.36-7.52 (m, 4H), 7.62-7.64 (m, 1H), 7.70-7.75 (m, 3H). ¹³C NMR (acetone-d₆): δ 106.08, 108.19, 121.37, 121.83, 123.63, 127.05, 127.96 (2C), 128.06, 129.68 (2C), 137.49, 139.25, 142.18, 162.05. MS m/z 253 (M⁺, 100%), 191 (M⁺-H₂O—CO₂, 65%), 190 (M⁺-H₂O—CO₂—H, 86%), 165 (M⁺-H₂O—CO₂—C₂H₂, 32%). HPLC, t_{R} 9.2 min.

### Example 33

¹H NMR (acetone-d₆): δ 7.29 (d, 1H, J=0.7 Hz), 7.61 (dt, 1H, J=8.8, 0.7 Hz), 8.03 (dd, 1H, J=8.8, 1.5 Hz), 8.47 (dd, 1H, J=1.5, 0.7 Hz). MS m/z 221 (M⁺, 78%), 205 (M⁺-O, 100%), 133 (M⁺-2CO₂, 57%).

### Example 34

¹H NMR (DMSO-d₆): δ 6.97 (ddd, 1H, J=9.7, 8.8, 2.4 Hz), 7.04 (d, 1H, J=0.7), 7.18 (ddd, 1H, J=9.9, 1.8, 0.9 Hz), 7.67 (ddd, 1H, J=8.6, 5.3, 0.4 Hz). MS m/z 195 (M⁺, 100%), 177 (M⁺-H₂O, 43%), 133 (M⁺-CO₂—H₂O, 72%).

### Example 35

¹H NMR (DMSO-d₆): δ 7.15 (s, 1H), 7.57-7.61 (m, 2H), 8.24 (s, 1H).

### Example 36

¹H NMR (DMSO-d₆): δ 7.12 (d, 1H, J=0.9 Hz), 7.41 (dd, 1H, J=8.2, 1.5 Hz), 7.83 (dd, 1H, J=8.2, 0.7 Hz), 7.97 (dt, 1H, J=1.5, 0.7 Hz).

### Example 37

¹H NMR (DMSO-d₆): δ 6.88 (ddd, 1H, J=10.6, 5.1, 3.3 Hz), 7.01 (s, 1H), 7.26-7.31 (m, 2H). MS m/z 195 (M⁺, 100%), 133 (M⁺-OH—COOH, 21%).

### Example 38

¹H NMR (DMSO-d₆): δ 6.99 (qd, 1H, J=1.7, 0.8 Hz), 7.43-7.53 (m, 2H), 7.75-7.80 (m, 1H). ¹³C NMR (acetone-d₆) δ 103.60, 114.89, 117.91 (q, J=3.0 Hz), 119.40 (q, J=5.2 Hz), 123.27 (q, J=34.8 Hz), 125.16, 125.51 (q, J=260.9 Hz), 128.31, 136.96, 161.39. MS m/z 245 (M⁺, 100%), 229 (M⁺-O, 9%), 183 (M⁺-H₂O—CO₂, 33%). HPLC, t_{R} 8.8 min.

### Example 39

¹H NMR (DMSO-d₆): δ 7.01 (s, 1H), 7.41-7.61 (m, 7H).

### Example 40

¹H NMR (DMSO-d₆): δ 7.03 (d, 1H, J=0.7 Hz), 7.19 (dd, 1H, J=6.6, 1.7 Hz), 7.37-7.57 (m, 5H), 7.64-7.69 (m, 2H). ¹³C NMR (acetone-d₆): δ 105.31, 109.61, 120.50, 121.17, 126.43, 126.73, 128.29, 129.37 (2C), 129.55 (2C), 136.65, 137.43, 140.67, 162.01. MS m/z 253 (M⁺, 100%), 237 (M⁺-O, 8%), 191 (M⁺-H₂O—CO₂, 25%), 190 (M⁺-H₂O—CO₂—H, 62%), 165 (M⁺-H₂O—CO₂—C₂H₂, 62%). HPLC, t_{R} 8.9 min.

### Example 41

¹H NMR (DMSO-d₆): δ 0.93 (t, 3H, J=7.2 Hz), 1.39 (sext., 2H, J=7.3 Hz), 1.69 (quint., 2H, J=7.5 Hz), 2.75 (t, 2H, J=7.6 Hz), 7.32 (d, 1H, J=0.7 Hz), 7.42-7.53 (m, 2H), 7.57 (ddd, 1H, J=7.1, 2.2, 0.6 Hz), 8.62 (s, 1H). ¹³C NMR (DMSO-d₆): δ 13.76, 21.77, 24.68, 31.00, 103.41, 110.08, 112.48, 113.30, 121.29, 124.86, 127.68, 129.96, 137.01, 147.53, 160.73. MS m/z 301 (M+H⁺), 285 (M+H⁺—O).

### Example 42

¹H NMR (DMSO-d₆): δ 2.87 (t, 2H, J=6.4 Hz), 3.72 (t, 2H, J=6.9 Hz), 7.11 (d, 1H, J=0.8 Hz), 7.65 (dd, 1H, J=8.6, 2.0 Hz), 7.83 (d, 1H, J=8.8 Hz), 7.89 (m, 1H), 8.69 (s, 1H). ¹³C NMR (DMSO-d₆): δ 29.27, 60.20, 100.48, 104.87, 110.77, 113.15, 120.49, 120.89, 123.70, 128.85, 135.53, 145.53, 160.80. MS m/z 288 (M⁺).

### Example 43

¹H NMR (DMSO-d₆): δ 2.86 (t, 2H, J=6.9 Hz), 3.71 (t, 2H, J=6.9 Hz), 7.12 (s, 1H), 7.61 (d, 1H, J=9.0 Hz), 7.80 (dd, 1H, J=9.0, 1.9 Hz), 8.10 (d, 1H, J=1.7 Hz), 8.52 (s, 1H). MS m/z 288 (M⁺47%), 272 (M⁺-O, 50%), 226 (M⁺-C₂H₅O, —OH 52%), 181 (M⁺-OH, —COOH, —C₂H₅O, 100%).

### Example 44

¹H NMR (CD₃OD): δ 1.12-1.33 (m, 4H), 3.11-3.24 (m, 1H), 7.23 (s, 1H), 7.58 (dd, 1H, J=8.8, 0.7 Hz), 7.87 (dd, 1H, J=8.8, 1.2 Hz), 8.28 (dd, 1H, J=1.4, 0.7 Hz). ¹³C NMR (CD₃OD): δ 6.38 (2C), 32.09, 107.83, 110.80 (2C), 122.12, 125.38, 125.60 (2C), 139.31, 163.15, 168.90. MS m/z 324 (M⁺5%), 322 (M⁺-H₂, 100%), 279 (M⁺-COOH, 18%).

### Example 45

¹H NMR (CD₃OD): δ 1.12-1.19 (m, 2H); 1.29-1.34 (m, 2H); 3.14-3.25 (m, 1H); 7.12 (t, 1H, J=0.7 Hz); 7.62 (ddd, 1H, J=8.4, 1.6, 0.7 Hz); 7.74 (d, 1H, J=8.4 Hz); 8.13 (dd, 1H, J=1.6, 0.8 Hz). ¹³C NMR (CD₃OD): δ 6.36 (2C), 32.03, 105.94, 111.93 (2C), 120.80, 123.51 (2C), 129.33, 136.58, 163.20, 168.83.

### Example 46

¹H NMR (DMSO-d₆): δ 7.11 (d, 1H, J=0.9 Hz), 7.79 (dd, 1H, J=7.8, 1.5 Hz), 7.86 (d, 1H, J=7.8 Hz), 8.17-8.19 (m, 1H). ¹³C NMR (DMSO-d₆): δ 104.63, 108.58, 118.82, 122.66, 123.28, 128.85, 135.40, 160.84. HPLC, t_{R} 1.4 min.

### Example 47

¹H NMR (acetone-d₆) δ (ppm): 2.67 (t, 2H, J=7.8 Hz), 2.97 (t, 2H, J=7.6 Hz), 7.17 (s, 1H), 7.36 (d, 2H, J=8.0 Hz), 7.58-7.69 (m, 4H), 7.91-7.92 (m, 1H), 10.85 (bs, 1H). ¹³C NMR (acetone-d₆): δ 31.15, 35.85, 106.57, 110.81, 120.97, 122.83, 125.80, 127.78 (2C), 129.64 (2C), 134.77, 140.27, 140.46, 161.85, 173.78. MS m/z 325 (M⁺, 12%); 255 (M⁺-C₃H₂O₂, 100%); 175 (M⁺-C₉H₁₀O₂, 18%); 149 (M⁺-C₉H₆O₃N, 31%).

### Example 48

¹H NMR (DMSO-d₆): δ 7.41 (s, 1H), 7.48-7.69 (m, 4H), 7.96 (dt, 1H, J=8.0, 1.4 Hz), 8.27 (dt, 1H, J=7.6, 1.5 Hz), 8.61 (t, 1H, J=1.6 Hz), 9.50 (s, 1H). ¹³C NMR (DMSO-d₆): δ 103.27, 110.53, 112.86, 113.35, 121.33, 124.81, 126.14, 127.88, 128.89, 129.27, 129.60, 129.69, 130.60, 131.51, 136.97, 145.95, 160.73, 167.00. MS m/z 365 (M+H⁺).

### Example 49

¹H NMR (acetone-d₆) δ (ppm): 2.68 (t, 2H, J=7.2 Hz), 2.99 (t, 2H, J=7.5 Hz), 7.13 (d, 1H, J=0.9 Hz), 7.39 (AA′/XX′, 2H, J_{AX}=8.1 Hz, J_{AA′/XX′}=2.0 Hz), 7.45 (dd, 1H, J=8.8, 1.5 Hz), 7.68 (AA′/XX′, 2H, J_{AX}=8.2 Hz, J_{AA′/XX′}=1.9 Hz), 7.71-7.77 (m, 2H). ¹³C NMR (acetone-d₆): δ 32.44, 35.81, 106.13, 107.99, 121.34, 123.59, 127.64, 127.96 (2C), 129.75 (2C), 131.61, 131.90, 139.16, 140.04, 141.17, 160.83, 173.76. MS m/z 325 (M⁺, 14%); 255 (M⁺-C₃H₂O₂, 32%); 175 (M⁺-C₉H₁₀O₂,16%); 149 (M⁺-C₉H₆O₃N, 100%).

### Example 50

¹H NMR (DMSO-d₆): δ 7.11 (s, 1H), 7.74 (dd, 1H, J=8.6, 1.5 Hz), 7.89 (d, 1H, J=8.8 Hz), 8.02-8.10 (m, 5H), 9.60 (s, 1H).

### Example 51

¹H NMR (acetone-d₆) δ (ppm): 6.98 (AA′/XX′, 2H, J_{AX}=9.1 Hz, J_{AA′/XX′}=2.8 Hz), 7.10 (d, 1H, J=0.9 Hz), 7.14 (dd, 1H, J=9.0, 2.2 Hz), 7.33 (d, 1H, J=2.2 Hz), 7.36 (AA′/XX′, 2H, J_{AX}=9.0 Hz, J_{AA′/XX′}=2.8 Hz), 7.59 (dt, 1H, J=9.0, 0.8 Hz).

### Example 52

¹H NMR (DMSO-d₆): δ 0.93 (t, 3H, J=7.3 Hz), 1.38 (sest., 2H, J=7.5 Hz), 1.66 (quint., 2H, J=7.5 Hz), 2.70 (t, 2H, J=7.6 Hz), 7.12 (s, 1H), 7.61 (d, 1H, J=9.0 Hz), 7.81 (dd, 1H, J=9.1, 1.9 Hz), 8.11 (d, 1H, J=1.6 Hz), 8.53 (s, 1H).

### Example 53

¹H NMR (DMSO-d₆): δ 7.20 (d, 1H, J=1.8 Hz), 7.38-7.43 (m, 1H), 7.71 (dd, 1H, J=8.6, 2.0 Hz), 7.88 (d, 1H, J=8.8 Hz), 7.96 (t, 1H, J=8.1 Hz), 8.02 (s, 1H), 8.11-8.16 (m, 1H), 8.63-8.69 (m, 1H), 9.31 (s, 1H), 12.17 (bs, 1H). MS m/z 322 (M+H⁺100%), 295 (M⁺-HCN, 60%).

### Example 54

¹H NMR (CD₃OD): δ 7.10 (d, 1H, J=0.6 Hz), 7.44 (dd, 1H, J=8.4, 1.6 Hz), 7.71 (d, 1H, J=8.4 Hz), 7.76-7.78 (m, 1H), 7.81 (AA′/XX′, 2H, J_{AX}=8.4 Hz, J_{AA′/XX′}=2.2 Hz), 8.11 (AA′/XX′, 2H, J_{AX}=8.6 Hz, J_{AA′/XX′}=2.4 Hz).

### Example 55

¹H NMR (DMSO-d₆): δ 7.22 (d, 1H, J=0.9 Hz), 7.66 (dd, 1H, J=8.4, 1.6 Hz), 7.88 (d, 1H, J=8.4 Hz), 8.10 (s, 1H). ¹³C NMR (DMSO-d₆): δ 106.15, 109.19, 118.55, 120.84, 124.29, 124.69, 129.13, 131.70, 158.50, 160.20, 161.59.

### Example 56

¹H NMR (acetone-d₆) δ (ppm): 7.18 (d, 1H, J=0.9 Hz), 7.58 (td, 1H, J=7.5, 0.4 Hz), 7.62 (dt, 1H, J=8.8, 0.7 Hz), 7.71 (dd, 1H, J=8.6, 1.6 Hz), 7.91 (dd, 1H, J=2.0, 1.3 Hz), 7.94-8.00 (m, 2H), 8.31 (t, 1H, J=1.6 Hz).

### Example 57

¹H NMR (acetone-d₆): δ 7.06-7.28 (m, 11H), 7.54 (s, 1H), 7.70 (s, 1H). ¹³C NMR (acetone-d₆): δ 106.28, 111.80, 121.88, 124.85, 126.80, 127.18, 127.51, 128.49, 128.56, 130.73, 130.84, 135.32, 136.47, 139.65, 142.93, 143.02, 162.01.

### Example 58

¹H NMR (CDCl₃): δ 2.31 (s, 3H), 3.21 (s, 3H), 7.02 (AA′XX′, 2H, J_{AX}=8.6 Hz, J_{AA′/XX′}=2.1 Hz), 7.08-7.18 (m, 2H), 7.23 (d, 1H, J=1 Hz), 7.24 (dd, 1H, J=8.4 Hz, 1.6 Hz), 7.78 (dt, 1H, J=1.8 Hz, 0.8 Hz), 7.83 (dd, 1H, 8.4 Hz, 0.8 Hz). ¹³C NMR (CDCl₃): δ 20.97, 38.67, 105.88, 111.21, 120.01, 123.648, 124.91, 127.22, 130.06, 134.06, 135.25, 137.67, 140.14, 161.27. MS m/z 359 (M⁺-H).

### Example 59

¹H NMR (acetone-d₆): δ 3.29 (s, 3H), 7.20 (dd, 1H, J=8.4 Hz, 1.8 Hz), 7.21 (d, 1H, J=1.8 Hz), 7.40-7.50 (m, 2H), 7.65-7.74 (m, 2H), 7.78-7.86 (m, 2H). ¹³C NMR (acetone-d₆): δ 38.12, 105.71, 111.18, 119.61, 123.96, 125.16, 126.62 (q, 2C, J=3.7 Hz), 127.13 (2C), 127.75, 128.66 (q, J=33.0 Hz), 130.20, 132.38 (q, J=269.8 Hz), 133.39, 146.30, 161.38. MS m/z 415 (M+H⁺, 5%), 239 (CF₃PhN(Me)SO₂+H⁺, 20%), 177 (M+H⁺—CF₃PhN(Me)SO₂, 100%).

### Example 60

¹H NMR (acetone-d₆): δ 3.22 (s, 3H), 7.04-7.19 (m, 4H), 7.22 (d, 1H, J=0.9 Hz), 7.23 (dd, 1H, J=8.4, 1.6 Hz), 7.73-7.76 (m, 1H), 7.84 (dd, 1H, J=8.6, 0.8 Hz). ¹³C NMR (acetone-d₆): δ 38.71, 105.95, 111.25, 116.19 (d, 2C, J=22.9 Hz), 119.95, 123.78, 125.05, 129.48 (d, 2C, J=9.2 Hz), 130.08, 133.55, 135.32, 138.89 (d, J=3.7 Hz), 161.16, 162.13 (d, J=244.4 Hz).

### Example 61

¹H NMR (acetone-d₆): δ 3.22 (s, 3H), 7.14-7.25 (m, 4H), 7.36 (AA′XX′, 2H, J_{AX}=9.0 Hz, J_{AA′/XX′}=2.4 Hz), 7.77 (pseudo-t, 1H, J=0.8 Hz), 7.83 (dd, 1H, J=8.4, 0.4 Hz). ¹³C NMR (acetone-d₆): 38.69, 105.99, 111.49, 120.08, 124.09, 125.16, 129.09, 129.82, 132.55, 133.17, 133.66, 135.41, 141.86, 161.56. MS m/z 403 (M+Na⁺, 9%), 370 (M+Na⁺—O—OH, 100%).

### Example 62

¹H NMR (DMSO-d₆): δ 7.13 (s, 1H), 7.64 (t, 1H, J=7.7 Hz), 7.67 (dd, 1H, J=8.6, 1.8 Hz), 7.87 (d, 1H, J=2.0 Hz), 7.94 (dt, 1H, J=8.2, 1.4 Hz), 8.18-8.23 (m, 2H), 8.55 (t, 1H, J=1.6 Hz), 9.45 (s, 1H). ¹³C NMR (DMSO-d₆): 104.83, 110.73, 113.59, 117.82, 120.22, 120.60, 125.84, 128.58, 129.07, 129.21 (2C), 130.31, 130.69, 131.45, 134.91, 146.11, 160.62, 166.80.

### Example 63

¹H NMR (acetone-d₆): δ 7.17 (d, 1H, J=0.7 Hz), 7.52 (dd, 1H, J=8.4, 1.6 Hz), 7.81 (dd, 1H, J=8.4, 0.7 Hz), 7.82-7.90 (m, 3H), 7.96-8.03 (m, 2H). ¹³C NMR (acetone-d₆): δ 106.00, 108.83, 121.23, 122.45, 123.94, 125.48 (q, J=269.7 Hz), 126.54 (q, 2C, J=3.7 Hz), 127.60, 128.62 (2C), 129.35 (q, J=34.8 Hz), 137.36, 137.38, 146.10, 161.99. MS m/z 321 (M⁺, 100%), 305 (M⁺-O, 18%).

### Example 64

¹H NMR (acetone-d₆): δ 7.15 (d, 1H, J=0.6 Hz), 7.18-7.32 (m, 2H), 7.42 (dd, 1H, J=8.6, 1.6 Hz), 7.67-7.86 (m, 4H). ¹³C NMR (acetone-d₆): δ 106.19, 108.17, 116.32 (d, 2C, J=21.0 Hz), 121.26, 121.77, 123.68, 127.13, 129.79 (d, 2C, J=8.2 Hz), 137.50, 138.14, 138.52 (d, J=3.7 Hz), 161.90, 163.15 (d, J=244.5 Hz). MS m/z 271 (M⁺, 100%), 255 (M⁺-O, 33%), 208 (M⁺-CO₂—F, 55%).

### Example 65

¹H NMR (acetone-d₆): δ 7.17 (d, 1H, J=0.7 Hz), 7.18-7.28 (m, 2H), 7.56-7.63 (m, 4H), 7.91 (dd, 1H, J=1.5, 0.9 Hz). ¹³C NMR (DMSO-d₆): δ 101.65, 109.80, 115.48 (d, 2C, J=21.1 Hz), 119.71, 121.26, 122.95, 127.41, 128.39 (d, 2C, J=7.3 Hz), 130.96, 133.06, 137.65 (d, J=2.7 Hz), 161.21 (d, J=242.6 Hz), 162.50. MS m/z 271 (M⁺, 60%), 255 (M⁺-O, 100%), 208 (M⁺-CO₂—F, 88%).

### Example 66

¹H NMR (DMSO-d₆): δ 7.10 (s, 1H), 7.56 (d, 1H, J=8.6 Hz), 7.70 (dd, 1H, J=8.6, 1.6 Hz), 7.80 (d, 2H, J=8.4 Hz), 7.92 (d, 2H, J=8.2 Hz), 8.02 (s, 1H). ¹³C NMR (DMSO-d₆): δ 105.11, 110.31, 120.67, 121.42, 124.10, 124.41 (q, J=271.0 Hz), 125.61 (q, 2C, J=3.6 Hz), 126.93 (q, J=30.7 Hz), 127.25 (2C), 127.59, 131.02, 135.62, 144.84, 161.00.

### Example 67

¹H NMR (DMSO-d₆): δ 6.07 (s, 2H), 7.01 (d, 1H J=8.1 Hz), 7.02 (s, 1H), 7.19 (dd, 1H, J=8.5, 1.3 Hz), 7.29 (d, 1H, J=1.5 Hz), 7.36 (dd, 1H, J=8.6, 1.5 Hz), 7.55 (m, 1H), 7.67 (d, 1H, J=8.8 Hz). ¹³C NMR (DMSO-d₆): δ 101.14, 104.65, 106.78, 107.29, 108.69, 119.98, 120.11, 120.46, 122.53, 127.16, 134.97, 136.50, 136.86, 146.69, 147.95, 161.13.

### Example 68

¹H NMR (DMSO-d₆): δ 3.79 (s, 3H), 6.96-7.08 (m, 3H), 7.47 (d, 1H, J=8.6 Hz), 7.52-7.66 (m, 3H), 7.83 (s, 1H). ¹³C NMR (DMSO-d₆): δ 55.16, 104.90, 110.06, 114.28, 119.20, 121.53, 124.10, 127.19, 127.70, 132.58, 133.31, 135.11, 158.27, 161.20. MS m/z 284 (M+H⁺, 20%), 283 (M⁺, 100%), 267 (M⁺-O, 99%), 252 (M⁺-CH₃O, 19%).

### Example 69

¹H NMR (acetone-d₆): δ 3.24 (s, 3H), 7.11-7.16 (m, 1H), 7.26 (d, 1H, J=0.9 Hz), 7.31 (td, 1H, J=7.4 1.8 Hz), 7.39 (td, 1H, J=7.3, 1.8 Hz), 7.50 (dd, 1H, J=8.4, 1.6 Hz), 7.51-7.55 (m, 1H), 7.91 (dd, 1H, J=8.6, 0.7 Hz), 7.95 (dt, 1H, J=1.6, 0.8 Hz), 10.80 (bs, 1H). ¹³C NMR (acetone-d₆): 38.78, 105.99, 111.16, 119.95, 124.05, 125.01, 128.56, 130.55, 131.33 (2C), 135.10, 135.43, 136.16, 138.21, 139.74, 161.19. MS m/z 380 (M⁺, 20%), 268 (M⁺-C₆H₅Cl), 240 (M⁺-oClPhNMe). HPLC, t_{R}=9.4 min.

### Example 70

¹H NMR (DMSO-d₆): δ 7.04 (d, 1H, J=0.7 Hz), 7.41 (dd, 1H, J=8.3, 1.0 Hz), 7.49 (d, 1H, J=8.4 Hz), 7.57 (dd, 1H, J=8.4, 1.1 Hz), 7.66 (d, 1H, J=0.9 Hz), 7.72 (d, 1H, J=8.4 Hz), 7.82 (d, 1H, J=1.6 Hz). ¹³C NMR (DMSO-d₆): 104.58, 107.53, 108.89, 110.35, 120.04, 120.56, 122.73, 123.11, 127.54, 131.23 (t, J=262 Hz), 135.66, 136.33, 137.81, 142.05, 143.43, 161.06. MS m/z 333 (M⁺, 26%), 317 (M⁺-O, 12%), 289 (M⁺-CO₂, 5%), 271 (M⁺-CO₂—H₂O, 7%), 245 (M⁺-CO₂—H₂O—C₂H₂, 14%), 177 (M⁺-C₇H₃F₂O₂+H, 100%). HPLC, t_{R}=10.5 min.

### Example 71

¹H NMR (DMSO-d₆): δ 7.07 (s, 1H), 7.44-7.56 (m, 3H), 7.63 (dd, 1H, J=8.7, 1.6 Hz), 7.71 (AA′/XX′, 2H, J_{AX}=8.6 Hz, J_{AA′/XX′}=1.5 Hz), 7.93 (d, 1H, J=0.8 Hz). ¹³C NMR (DMSO-d₆): δ 105.11, 110.24, 120.07, 121.44, 124.08, 127.43, 128.38 (2C), 128.78 (2C), 131.42, 135.46, 139.70, 161.10. MS m/z 289 (³⁷Cl: M⁺, 40%), 287 (³⁵Cl: M⁺, 100%), 271 (³⁵Cl: M⁺-O, 85%). HPLC, t_{R}=9.9 min.

### Example 72

¹H NMR (DMSO-d₆): δ 7.04 (d, 1H, J=0.8 Hz), 7.41 (dd, 1H, J=8.4, 1.4 Hz), 7.52 (AA′XX′, 2H, J_{AX}=8.4 Hz, J_{AA′/XX′}=2.0 Hz), 7.65 (s, 1H), 7.68-7.82 (m, 3H). ¹³C NMR (DMSO-d₆): δ 104.49, 107.16, 119.71, 120.58, 122.77, 127.52, 128.58 (2C), 128.85 (2C), 132.04, 135.53, 136.31, 139.39, 161.08. MS m/z 289 (³⁷Cl: M⁺, 15%), 287 (³⁵Cl: M⁺, 30%), 271 (³⁵Cl: M⁺-O, 55%), 190 (³⁵Cl: M⁺-Cl—H₂O—CO₂, 100%). HPLC, t_{R}=10.2 min.

### Example 73

¹H NMR (DMSO-d₆): δ 7.02-7.22 (m, 11H), 7.41 (d, 1H, J=0.8 Hz). ¹³C NMR (DMSO-d₆): δ 101.08, 116.65, 119.73, 120.40 (q, J=4.0 Hz), 123.92 (q, J=32.3 Hz), 124.09 (q, J=269.3 Hz), 126.48, 126.70 (2C), 127.59 (2C), 128.67, 129.36, 129.87 (2C), 130.85 (2C), 133.20, 135.62, 137.12, 140.06, 160.60. HPLC, t_{R}=11.2 min.

### Example 74

¹H NMR (acetone-d₆): δ 0.86 (t, 3H, J=7.0 Hz), 1.35-1.42 (m, 4H), 3.66 (t, 2H, J=6.4 Hz), 7.08-7.13 (m, 2H), 7.22 (d, 1H, J=0.7 Hz), 7.28-7.37 (m, 4H), 7.77 (s, 1H), 7.83 (d, 1H, J=8.6 Hz). ¹³C NMR (acetone-d₆): δ 13.84, 20.15, 50.71, 105.91, 110.99, 119.82, 123.74, 124.80, 128.44, 129.62, 129.86, 135.30, 135.78, 140.24, 161.28. HPLC, t_{R}=10.1 min.

### Example 75

¹H NMR (DMSO-d₆): δ 2.66 (s, 6H), 7.06 (d, 1H, J=1.2 Hz), 7.51 (dd, 1H, J=8.6, 1.6 Hz), 7.76-7.85 (m, 4H), 8.02 (AA′XX′, 2H, J_{AX}=8.8 Hz, J_{AA′/XX′}=1.4 Hz). ¹³C NMR (DMSO-d₆): δ 37.64 (2C), 104.40, 107.87, 119.84, 121.11, 122.95, 127.58 (2C), 127.90, 128.21 (2C), 133.09, 134.88, 136.21, 144.87, 161.04. HPLC, t_{R}=8.5 min.

### Example 76

¹H NMR (acetone-d₆): δ 7.01 (dd, 1H, J=1.8, 0.9 Hz), 7.10 (d, 1H, J=0.9 Hz), 7.42 (dd, 1H, J=8.4, 1.5 Hz), 7.65-7.72 (m, 3H), 8.13 (dd, 1H, J=1.5, 0.9 Hz). ¹³C NMR (acetone-d₆): δ 106.24, 106.75, 109.61, 120.33, 121.52, 123.59, 127.75, 129.64, 130.64, 137.34, 140.02, 144.86, 162.14. MS m/z 243 (M⁺, 56%), 227 (M⁺-O, 100%), 180 (M⁺-CO₂—H₂O, 26%). HPLC, t_{R}=8.6 min.

### Example 77

¹H NMR (acetone-d₆): δ 7.16 (d, 1H, J=0.7 Hz), 7.31-7.38 (m, 1H), 7.49 (dd, 1H, J=8.4, 1.6 Hz), 7.63 (t, 1H, J=7.9 Hz), 7.68-7.70 (m, 1H), 7.77-7.83 (m, 3H). ¹³C NMR (acetone-d₆): δ 105.95, 108.72, 120.34, 120.55, 121.23, 121.62 (q, J=253.4 Hz), 122.41, 123.94, 126.94, 127.53, 131.48, 137.31, 137.49, 144.81, 150.62, 162.27. MS m/z 337 (M⁺, 56%), 321 (M⁺-O, 63%), 293 (M⁺-CO₂, 5%), 275 (M⁺-CO₂—H₂O, 8%), 249 (M⁺-CO₂—H₂O—C₂H₂, 13%), 190 (M⁺-C₆H₄F₃O+H, 100%), 177 (M⁺-C₇H₄F₃O+H, 20%). HPLC, t_{R}=10.4 min.

### Example 78

¹H NMR (DMSO-d₆): δ 7.00 (qd, 1H, J=1.8, 0.7 Hz), 7.55 (d, 2H, J=8.4 Hz), 7.76 (s, 1H), 7.84 (d, 2H, J=8.6 Hz), 7.98 (s, 1H). ¹³C NMR (DMSO-d₆): δ 101.19, 111.55, 115.81, 115.83, 117.43 (q, J=5.5 Hz), 121.93 (q, J=33.0 Hz), 124.38 (q, J=271.9 Hz), 128.87 (2C), 128.98 (2C), 132.73, 134.82, 136.11, 137.92, 160.68. HPLC, t_{R}=11.0 min.

### Example 79

¹H NMR (acetone-d₅): δ 7.16 (d, 1H, J=0.7 Hz), 7.34-7.56 (m, 3H), 7.72-7.90 (m, 9H). ¹³C NMR (acetone-d₆): δ 106.10, 108.23, 121.37, 122.12, 123.78, 126.29, 127.65 (2C), 128.18, 128.26 (2C), 128.51 (2C), 129.77 (2C), 137.54, 138.85, 140.84, 141.33, 141.44, 162.40. HPLC, t_{R}=10.4 min.

### Example 80

¹H NMR (acetone-d₆): δ 2.67 (q, 3H, J=1.8 Hz), 7.38-7.58 (m, 3H), 7.78-7.84 (m, 3H), 8.03 (dq, 1H, J=1.5, 0.7 Hz). ¹³C NMR (DMSO-d₆): δ 10.63 (q, J=5.2 Hz), 111.43, 111.63, 116.18, 117.76 (q, J=4.6 Hz), 121.60 (q, J=32.0 Hz), 124.37 (q, J=269.9 Hz), 126.96 (2C), 127.67, 127.88, 129.16 (2C), 135.50, 136.50, 139.08, 162.02. MS m/z 335 (M⁺, 18%), 320 (M⁺-CH₃, 18%), 319 (M⁺-O, 100%), 318 (M⁺-OH, 6%), 291 (M⁺-CO₂, 5%), 275 (M⁺-CO₂—H₂O, 46%).

### Example 81

¹H NMR (acetone-d₆): δ 7.16 (d, 1H, J=0.9 Hz), 7.42-7.50 (m, 3H), 7.75-7.80 (m, 2H), 7.88 (AA′XX′, 2H, J_{AX}=8.9 Hz, J_{AA′/XX′}=2.6 Hz). ¹³C NMR (acetone-d₆): δ 106.02, 108.48, 121.23, 121.43 (q, J=256.5 Hz), 122.06, 122.23 (2C), 123.81, 127.34, 129.66 (2C), 137.36, 137.60, 141.42, 149.21, 161.98.

### Example 82

¹H NMR (acetone-d₆): δ 3.25 (s, 3H), 7.16-7.22 (m, 3H), 7.30-7.39 (m, 3H), 7.52 (dd, 1H, J=8.3, 1.6 Hz), 7.64 (AA′XX′, 2H, J_{AX}=8.4 Hz, J_{AA′/XX′}=1.9 Hz), 7.81 (d, 1H, J=8.6 Hz), 7.85-7.87 (m, 1H), 7.95 (AA′XX′, 2H, J_{AX}=8.4 Hz, J_{AA′/XX′}=1.8 Hz). ¹³C NMR (acetone-d₆): δ 38.67, 106.02, 109.04, 121.28, 122.72, 124.01, 127.42 (2C), 127.98, 128.35 (2C), 129.26 (2C), 129.68 (2C), 136.67, 137.23, 137.25, 142.89, 146.68, 162.21.

### Example 83

¹H NMR (acetone-d₆): δ 2.67 (q, 3H, J=1.6 Hz), 7.55 (AA′XX′, 2H, J_{AX}=8.6 Hz, J_{AA′/XX′}=2.4 Hz), 7.81 (s, 1H), 7.85 (AA′XX′, 2H, J_{AX}=8.8 Hz, J_{AA′/XX′}=2.2 Hz), 8.04 (s, 1H). ¹³C NMR (acetone-d₆): δ 11.24 (q, J=5.5 Hz), 112.62, 114.86, 117.81, 119.04 (q, J=6.4 Hz), 123.63 (q, J=33.6 Hz), 125.35 (q, J=271.0 Hz), 127.12, 129.63 (2C), 129.89 (2C), 134.27, 136.37, 137.61, 139.39, 163.04. HPLC, t_{R} 11.6 min.

### Example 84

¹H NMR (DMSO-d₆): δ 7.11 (d, 1H, J=0.9 Hz), 7.21 (dd, 1H, J=8.2, 1.6 Hz), 7.45-7.64 (m, 5H), 7.77 (dd, 1H, J=8.2, 0.6 Hz), 7.86 (dd, 1H, J=7.9, 1.3 Hz), 7.97 (d, 1H, J=8.6 Hz), 8.02 (dd, 1H, J=7.9, 1.6 Hz). ¹³C NMR (DMSO-d₆): δ 104.65, 110.28, 120.26, 121.97, 122.84, 125.30, 125.50, 125.83, 126.24, 126.99, 127.34, 127.48, 128.28, 130.94, 133.38, 135.99, 136.66, 139.86, 161.06. HPLC, t_{R} 10.3 min.

### Example 85

¹H NMR (acetone-d₆): δ 7.17 (d, 1H, J=0.9 Hz), 7.51-7.57 (m, 2H), 7.63 (dd, 1H, J=8.5, 1.6 Hz), 7.81 (dd, 1H, J=8.4, 0.6 Hz), 7.92-7.97 (m, 3H), 7.99-8.06 (m, 2H), 8.29 (d, 1H, J=1.5 Hz). ¹³C NMR (DMSO-d₆): δ 104.52, 107.40, 120.09, 120.40, 120.49, 122.68, 125.23 (2C), 125.95, 126.30, 127.37, 128.14, 128.39, 132.09, 133.35, 136.50, 136.68, 137.86, 161.08. HPLC, t_{R}=10.1 min.

### Example 86

¹H NMR (acetone-d₆): δ 7.20 (qd, 1H, J=1.6, 0.8 Hz), 7.53 (dd, 1H, J=8.6, 2.0 Hz), 7.58-7.63 (m, 2H), 7.68 (d, 1H, J=1.8 Hz), 7.89 (s, 1H). ¹³C NMR (acetone-d₆): δ 103.06, 115.42, 117.49, 120.90 (q, J=4.8 Hz), 123.10 (q, J=32.5 Hz), 125.41 (q, J=272.9 Hz), 128.47, 129.17, 130.31, 133.79, 133.92, 134.79, 135.05, 136.45, 139.07, 161.70. HPLC, t_{R}=11.9 min.

### Example 87

¹H NMR (acetone-d₆): δ 3.26 (s, 3H), 7.10-7.16 (m, 1H), 7.22 (dd, 1H, J=8.4, 1.6 Hz), 7.23 (d, 1H, J=0.9 Hz), 7.25-7.28 (m, 1H), 7.32-7.36 (m, 2H), 7.79-7.81 (m, 1H), 7.84 (dd, 1H, J=8.9, 0.6 Hz). ¹³C NMR (acetone-d₆): δ 38.34, 105.20, 111.25, 119.61, 123.76, 125.10, 125.34, 127.27, 127.74, 130.22, 130.88, 133.24, 134.37, 134.74, 144.17, 161.85.

### Example 88

¹H NMR (acetone-d₆): δ 3.20 (s, 3H), 7.10-7.15 (m, 2H), 7.26-7.33 (m, 4H), 7.42 (dd, 1H, J=8.9, 1.7 Hz), 7.63 (dt, 1H, J=9.0, 0.8 Hz), 7.99 (dd, 1H, J=1.6-0.7 Hz). ¹³C NMR (acetone-d₆): δ 38.47, 107.39, 110.63, 121.26, 124.52, 124.74, 127.25 (2C), 127.73, 128.64, 129.46 (2C), 129.93, 137.65, 142.91, 161.54. HPLC, t_{R}=8.9 min.

### Example 89

¹H NMR (CD₃OD): δ 7.36-7.49 (m, 2H), 7.67-7.70 (m, 2H), 8.65 (bs, 1H).

### Example 90

¹H NMR (acetone-d₆) δ (ppm): 6.60-6.90 (bm, 3H), 7.26 (bs, 1H), 11.64 (bs, 1H).

### Example 91

¹H NMR (CD₃OD); tautomer A: δ 7.35 (dd, 1H, J=8.6, 1.9 Hz), 7.55 (d, 1H, J=8.8 Hz), 8.33 (d, 1H, J=2.4 Hz); tautomer B: δ 7.28 (dd, 1H, J=8.6, 2.0 Hz), 7.61 (d, 1H, J=8.9 Hz), 7.64 (d, 1H, J=2.0 Hz).

### Example 92

¹H NMR (CD₃OD): δ 7.40-7.53 (m, 3H), 7.66-7.75 (m, 2H), 7.85-8.02 (m, 3H), 9.20 (bs, 1H).

### Example 93

¹H NMR (DMSO-d₆): δ 3.73 (s, 2H), 6.89 (s, 1H), 7.45-7.54 (m, 3H), 8.18-8.22 (m, 2H). ¹³C NMR (DMSO-d₆): δ 32.42, 104.12, 128.08 (2C), 128.72 (2C), 131.42, 132.38, 136.37, 160.44, 169.78, 171.84.

### Example 94

¹H NMR (DMSO-d₆): δ 2.25 (s, 3H), 3.74 (s, 2H), 7.27 (s, 1H). ¹³C NMR (DMSO-d₆): δ 22.46, 32.87, 108.06, 136.42, 141.85, 169.02, 169.51.

### Example 95

¹H NMR (DMSO-d₆): δ 3.74 (s, 2H), 6.93 (s, 1H), 8.04 (d, 2H, J=8.3 Hz), 8.30 (d, 2H, J=8.4 Hz). ¹³C NMR (DMSO-d₆): δ 32.33, 104.41, 128.79 (2C), 129.14 (2C), 132.36, 132.96, 140.36, 161.40, 166.92, 169.73, 171.29. MS m/z 322 (M⁺10%), 230 (M⁺-CO₂, —CH₂, —OH, —OH 38%), 215 (M⁺-COOH, —COOH, —OH 100%).

### Example 96

¹H NMR (DMSO-d₆): δ 3.74 (s, 2H), 6.92 (s, 1H), 7.62 (t, 1H, J=7.7 Hz), 8.08 (dt, 1H, J=7.8, 1.6 Hz), 8.40 (dt, 1H, J=7.7, 1.5 Hz), 8.81 (t, 1H, J=1.6 Hz). ¹³C NMR (DMSO-d₆): δ 32.29, 104.32, 129.10, 129.63, 129.89, 130.72, 131.12, 132.34, 133.33, 136.95, 166.48, 166.98, 169.71.

Biologic Assays: Determination of the Enzyme Inhibition of Isoform 5 (LDH5, LDH-A) and Isoform 1 (LDH1, LDH-B) of Human Lactate Dehydrogenases.

Compounds described in Examples 1-96 were evaluated in enzyme kinetic assays, in orded to assess their inhibitory properties on two human isoforms of lactate dehydrogenase (LDH): hLDH5, which contains exclusively the LDH-A subunit (Lee Biosolution Inc., USA); hLDH1, which contains instead only the LDH-B subunit (SigmaAldrich, USA), with the purpose to verify the isoform selectivities of these compounds.

The LDH reaction is carried out by following the “forward” direction (pyruvate→lactate). The kinetic parameters of the substrate (pyruvate) and the cofactor (NADH) are calculated by using a spectrophotometric measurement at the 340 nm wavelength, in order to monitor the rate of conversion of NADH into NAD⁺ at 37° C. and, therefore, the rate of progression is of the “forward” reaction. These assays were executed in small wells/cuvettes containing 1 mL of a solution composed of all the reagents dissolved in a pH 7.4 phosphate buffer (NaH₂PO₄/Na₂HPO₄).

The kinetic parameters for isoform hLDH1 relative to pyruvate are calculated by measuring the initial rate of reaction, using a 25-1000 μM range of pyruvate concentrations and a fixed 200 μM concentration of NADH. On the other hand, the kinetic parameters for the same isoform relative to NADH are instead calculated by measuring the initial rate of reaction, using a 12.5-200 μM range of NADH concentrations and a fixed 1000 μM concentration of pyruvate. All these assays are run with 0.005 U/mL di hLDH1.

The kinetic parameters for isoform hLDH5 relative to pyruvate are calculated by measuring the initial rate of reaction, using a 25-1000 μM range of pyruvate concentrations and a fixed 200 μM concentration of NADH. On the other hand, the kinetic parameters for the same isoform relative to NADH are instead calculated by measuring the initial rate of reaction, using a 12.5-200 μM range of NADH concentrations and a fixed 200 μM concentration of pyruvate. All these assays are run with 0.005 U/mL di hLDH5.

The resulting kinetic data (Michaelis-Menten constants) are determined by non-linear regression analysis. In a preliminary screening, the potential inhibition of either hLDH1 or hLDH5 is determined at a single maximal concentration of the inhibitor, that is, 100 μM of the compound in the pH 7.4 phosphate buffer solution containing 0.5% of DMSO. The compounds that turn out to be active are then submitted to further screening to evaluate their Kᵢ values. In particular, the apparent Kₘ′ values are evaluated in the presence of inhibitors (concentration range=1-100 μM). From the values of Kₘ′ so obtained, Kᵢ values for each single inhibitor are determined using double-reciprocal plots (Lineweaver-Burk).

Compounds repored in Examples 1-96 display one or more of the following features:
(i) an inhibitory activity against isoform hLDH5, which is competitive with cofactor NADH, with Kᵢ values in the 1-10000 μM range;
(ii) an inhibitory activity against isoform hLDH5, which is competitive with substrate pyruvate, with Kᵢ values in the 1-10000 μM range;
(iii) an inhibitory activity against isoform hLDH1, which is competitive with cofactor NADH, with Kᵢ values in the 90-10000 μM range.

1. Compounds, of general formula (I): 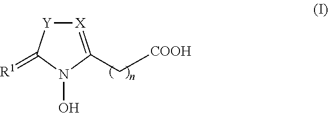
wherein:
n is selected from the group consisting of: 0, and 1;
X is selected from the group consisting of: N, N⁺—O⁻, and C—Z;
Y is selected from the group consisting of: S, O, and C═R²;
Z is selected from the group consisting of: hydrogen, OR^{A}, NR^{A}R^{B}, halogen, cyano, nitro, alkoxy, aryloxy, heteroaryloxy, —C(O)C₁₋₆-alkyl, —C(O)phenyl, —C(O)benzyl, —C(O)C₅₋₆-hetero cycle, —S—C₁₋₆-alkyl, —S-phenyl, —S-benzyl, —S—C₅₋₆-heterocycle, —S(O)C₁₋₆-alkyl, —S(O)phenyl, —S(O)benzyl, —S(O)C₅₋₆-heterocycle, —S(O)₂C₁₋₆-alkyl, —S(O)₂phenyl, —S(O)₂benzyl, —S(O)₂C₅₋₆-heterocycle, —S(O)₂NR^{A}R^{B}, C₁₋₆-alkyl, halo-C₁₋₆-alkyl, dihalo-C₁₋₆-alkyl, trihalo-C₁₋₆-alkyl, C₂₋₆-alkenyl, C₂₋₆-alkynyl, C₃₋₈-cycloalkyl, C₃₋₈-cycloalkyl-C₁₋₆-alkyl, phenyl, benzyl, and C₅₋₆-heterocycle;
R¹ is selected from the group consisting of: 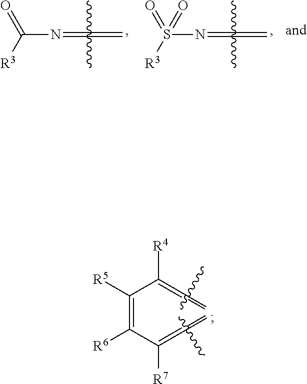
R² is selected, together with R¹, from: 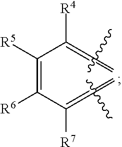
R³ is selected from the group consisting of: hydrogen, C₁₋₄-alkyl, halo-C₁₋₄-alkyl, dihalo-C₁₋₄-alkyl, trihalo-C₁₋₄-alkyl, C₂₋₆-alkenyl, C₂₋₄-alkynyl, C₃₋₆-cycloalkyl, C₃₋₆-cycloalkyl-C₁₋₂-alkyl, phenyl, benzyl, and C₅₋₆-heterocycle;
R⁴, R⁵, R⁶, and R⁷ are independently selected from the group consisting of: hydrogen, OR^{A}, NR^{A}R^{B}, —C(O)R^{A}, —C(O)OR^{A}—C(O)NR^{A}R^{B} halogen, cyano, nitro, alkoxy, aryloxy, heteroaryloxy, —C(O)C₁₋₆-alkyl, —C(O)phenyl, —C(O)benzyl, —C(O)C₅₋₆-heterocycle, —S—C₁₋₆-alkyl, —S-phenyl, —S-benzyl, —S—C₅₋₆-heterocycle, —S(O)C₁₋₆-alkyl, —S(O)phenyl, —S(O)benzyl, —S(O)C₅₋₆-heterocycle, —S(O)₂C₁₋₆-alkyl, —S(O)₂phenyl, —S(O)₂benzyl, —S(O)₂C₅₋₆-heterocycle, —S(O)₂NR^{A}R^{B}, C₁₋₆-alkyl, halo-C₁₋₆-alkyl, dihalo-C₁₋₆-alkyl, trihalo-C₁₋₆-alkyl, C₂₋₆-alkenyl, C₂₋₆-alkynyl, C₃₋₈-cycloalkyl, C₃₋₈-cycloalkyl-C₁₋₆-alkyl, phenyl, benzyl, naphthyl, and C₅₋₆-heterocycle;
wherein the phenyl, benzyl, naphthyl and C₅₋₆ heterocycle of the R³, R⁴, R⁵, R⁶, R⁷, R^{A} or R^{B} group may optionally be substituted with 1 to 3 groups independently selected from OR^{C}
wherein two OR^{C} groups may concur into forming a cycle, NR^{C}R^{D}, —C(O)R^{C}, —(C(O)OR^{C}, C₁₋₄-alkyl-OR^{C}, C₁₋₄-alkyl-C(O)OR^{C}, —C(O)NR^{C}R^{D}, —S(O)₂NR^{C}R^{D}, —S(O)₂C₁₋₆-alkyl, halogen, cyano, nitro, C₁₋₄-alkyl, halo-C₁₋₄-alkyl, dihalo-C₁₋₄-alkyl, trihalo-C₁₋₄-alkyl, aryl or heteroaryl, optionally substituted with C(O)OR^{C}; wherein any atom of the C₅-C₆ heterocycle of the R³, R⁴, R⁵, R⁶ and R⁷ group may be bound to an oxygen so to form an oxo or a a sulfoxo moiety;
wherein any alkyl, alkenyl and alkynyl groups of the R^{A}, R^{B}, R⁴, R⁵, R⁶ or R⁷ may optionally be substituted with 1-3 groups independently selected from OR^{C}, NR^{C}R^{D}, halogen, cyano and nitro; wherein any carbon-bound hydrogen atom may be substituted with a fluorine atom;
R^{A}, R^{B}, R^{C} and R^{D} being independently selected from the group consisting of: hydrogen, —C(O)C₁₋₆-alkyl, —C(O)phenyl, —C(O)benzyl, —C(O)C₅₋₆-heterocycle, —S(O)₂C₁₋₆-alkyl, —S(O)₂phenyl, —S(O)₂benzyl, —S(O)₂C₅₋₆-heterocycle, C₁₋₆-alkyl, halo-C₁₋₆-alkyl, dihalo-C₁₋₆-alkyl, trihalo-C₁₋₆-alkyl, C₂₋₆-alkenyl, C₂₋₆-alkynyl, C₃₋₈-cycloalkyl, C₃₋₈-cycloalkyl-C₁₋₆-alkyl, phenyl, benzyl, and C₅₋₆-heterocycle;
pharmaceutically acceptable salts, solvates, and physiologically functional derivatives thereof.

2. Compounds of formula (Ia): 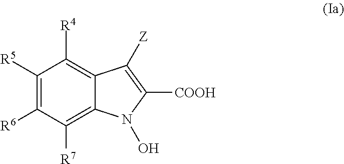
wherein Z, R⁴, R⁵, R⁶ and R⁷ are defined as in claim 1.

3. Compounds of formula (Ib): 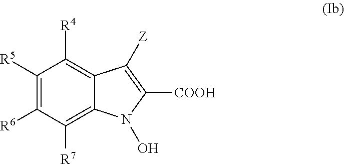
wherein Z is either H or a C₁₋₆ alkyl;
R⁴, R⁵, R⁶ and R⁷ are as defined in claim 1; and
such that at least one of R⁴, R⁵, R⁶ and R⁷ is selected from the group consisting of trihalo-C₁₋₄-alkyl, —S(O)₂NR^{A}R^{B}, phenyl, naphthyl and C₅₋₆ heterocycle, optionally substituted with 1 to 3 groups independently selected from the group consisting of OR^{C}, NR^{C}R^{D}, —C(O)R^{C}, —C(O)OR^{C}, C₁₋₄-alkyl-OR^{C}, C₁₋₄-alkyl-C(O)OR^{C}, —C(O)NR^{C}R^{D}, —S(O)₂NR^{C}R^{D}, —S(O)₂C₁₋₆-alkyl, halogen, cyano, nitro, C₁₋₄-alkyl, halo-C₁₋₄-alkyl, dihalo-C₁₋₄-alkyl, trihalo-C₁₋₄-alkyl, aryl and heteroaryl, optionally substituted with C(O)OR^{C}; and
R^{A}, R^{B}, R^{C} and R^{D} are as defined in claim 1.

4. (canceled)

5. A compound according to claim 2, selected from the group consisting of:
6-(3-carboxyphenyl)-1-hydroxy-1H-indol-2-carboxylic acid;
5-(4-carboxy-1H-1,2,3-triazol-1-yl)-1-hydroxy-1H-indol-2-carboxylic acid;
6-[4-(2-carboxyethyl)-1H-1,2,3-triazol-1-yl]-1-hydroxy-1H-indol-2-carboxylic acid;
1-hydroxy-6-phenyl-4-trifluoromethyl-1H-indol-2-carboxylic acid;
1-hydroxy-4-(4-phenyl-1H-1,2,3-triazol-1-yl)-1H-indol-2-carboxylic acid;
1-hydroxy-6-[N-methyl-N-phenylsulfamoyl]-1H-indol-2-carboxylic acid;
1-hydroxy-5-phenyl-1H-indol-2-carboxylic acid;
1-hydroxy-6-(4-methoxyphenyl)-1H-indol-2-carboxylic acid;
1-hydroxy-6-phenyl-1H-indol-2-carboxylic acid;
1-hydroxy-6-(2H-tetrazol-5-yl)-1H-indol-2-carboxylic acid;
5-[4-(2-carboxyethyl)phenyl]-1-hydroxy-1H-indol-2-carboxylic acid;
4-[4-(3-carboxyphenyl)-1H-1,2,3-triazol-1-yl]-1-hydroxy-1H-indol-2-carboxylic acid;
6-[4-(2-carboxyethyl)phenyl]-1-hydroxy-1H-indol-2-carboxylic acid;
6-[4-(4-carboxyphenyl)-1H-1,2,3-triazol-1-yl]-1-hydroxy-1H-indol-2-carboxylic acid;
5-(3-carboxyphenyl)-1-hydroxy-1H-indol-2-carboxylic acid;
1-hydroxy-5,6-diphenyl-1H-indole-2-carboxylic acid;
1-hydroxy-6-(N-methyl-N-p-tolylsulfamoyl)-1H-indole-2-carboxylic acid;
1-hydroxy-6-(N-methyl-N-(4-(trifluoromethyl)phenyl)sulfamoyl)-1H-indole-2-carboxylic acid;
6-(N-(4-fluorophenyl)-N-methylsulfamoyl)-1-hydroxy-1H-indole-2-carboxylic acid;
6-(N-(4-chlorophenyl)-N-methylsulfamoyl)-1-hydroxy-1H-indole-2-carboxylic acid;
5-(4-(3-carboxyphenyl)-1H-1,2,3-triazol-1-yl)-1-hydroxy-1H-indole-2-carboxylic acid;
1-hydroxy-6-(4-(trifluoromethyl)phenyl)-1H-indole-2-carboxylic acid;
6-(4-fluorophenyl)-1-hydroxy-1H-indole-2-carboxylic acid;
5-(4-fluorophenyl)-1-hydroxy-1H-indole-2-carboxylic acid;
1-hydroxy-5-(4-(trifluoromethyl)phenyl)-1H-indole-2-carboxylic acid;
6-(benzo[d][1,3]dioxol-5-yl)-1-hydroxy-1H-indole-2-carboxylic acid;
1-hydroxy-5-(4-methoxyphenyl)-1H-indole-2-carboxylic acid;
6-(N-(2-chlorophenyl)-N-methylsulfamoyl)-1-hydroxy-1H-indole-2-carboxylic acid;
6-(2,2-difluorobenzo[d][1,3]dioxol-5-yl)-1-hydroxy-1H-indole-2-carboxylic acid;
5-(4-chlorophenyl)-1-hydroxy-1H-indole-2-carboxylic acid;
6-(4-chlorophenyl)-1-hydroxy-1H-indole-2-carboxylic acid;
1-hydroxy-6,7-diphenyl-4-(trifluoromethyl)-1H-indole-2-carboxylic acid;
6-(N-butyl-N-phenylsulfamoyl)-1-hydroxy-1H-indole-2-carboxylic acid;
6-(4-(N,N-dimethylsulfamoyl)phenyl)-1-hydroxy-1H-indole-2-carboxylic acid;
6-(furan-3-yl)-1-hydroxy-1H-indole-2-carboxylic acid;
1-hydroxy-6-(3-(trifluoromethoxy)phenyl)-1H-indole-2-carboxylic acid;
6-(4-chlorophenyl)-1-hydroxy-4-(trifluoromethyl)-1H-indole-2-carboxylic acid;
6-(biphenyl-4-yl)-1-hydroxy-1H-indole-2-carboxylic acid;
1-hydroxy-3-methyl-6-phenyl-4-(trifluoromethyl)-1H-indole-2-carboxylic acid;
1-hydroxy-6-(4-(trifluoromethoxy)phenyl)-1H-indole-2-carboxylic acid;
1-hydroxy-6-(4-(N-methyl-N-phenylsulfamoyl)phenyl)-1H-indole-2-carboxylic acid;
6-(4-chlorophenyl)-1-hydroxy-3-methyl-4-(trifluoromethyl)-1H-indole-2-carboxylic acid;
1-hydroxy-6-(naphthalen-1-yl)-1H-indole-2-carboxylic acid;
1-hydroxy-6-(naphthalen-2-yl)-1H-indole-2-carboxylic acid;
6-(2,4-dichlorophenyl)-1-hydroxy-4-(trifluoromethyl)-1H-indole-2-carboxylic acid;
6-(N-(3-chlorophenyl)-N-methylsulfamoyl)-1-hydroxy-1H-indole-2-carboxylic acid;
1-hydroxy-5-(N-methyl-N-phenylsulfamoyl)-1H-indole-2-carboxylic acid;
pharmaceutically acceptable salts, solvates; and physiologically functional derivatives thereof.

6. A prodrug compound having formula (II) or (III) as follows: 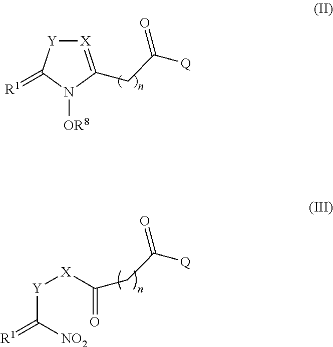
wherein Q is OR^{E}, SR^{E} or NR^{E}R^{F} where R^{E} and R^{F} are independently selected from the group consisting of: hydrogen, —C(O)C₁₋₆-alkyl, —C(O)phenyl, —C(O)benzyl, —C(O)C₅₋₆-heterocycle, —S(O)₂C₁₋₆-alkyl, —S(O)₂phenyl, —S(O)₂benzyl, —S(O)₂C₅₋₆-heterocycle, C₁₋₆-alkyl, halo-C₁₋₆-alkyl, dihalo-C₁₋₆-alkyl, trihalo-C₁₋₆-alkyl, C₂₋₆-alkenyl, C₂₋₆-alkynyl, C₃₋₈-cycloalkyl, C₃₋₈-cycloalkyl-C₁₋₆-alkyl, phenyl, benzyl, C₅₋₆-heterocycle, an L- or a D-sugar, a deoxysugar, a dideoxysugar, a glucose epimer, an (un)substituted sugar, a uronic acid or an oligosaccharide;
R⁸ is hydrogen, —C(O)C₁₋₆-alkyl, —C(O)phenyl, —C(O)benzyl, —C(O)C₅₋₆-heterocycle, trialkyl-silyl, dialkylaryl-silyl, C₁₋₄-alkyl, halo-C₁₋₄-alkyl, dialo-C₁₋₄-alkyl, trialo-C₁₋₄-alkyl, C₂₋₆-alkenyl, C₂₋₄-alkenyl, C₃₋₆-cycloalkyl, C₃₋₆-cycloalkyl-C₁₋₂-alkyl, phenyl, benzyl, C₅₋₆-heterocycle, an L- or a D-sugar, a deoxysugar, a dideoxysugar, a glucose epimer, an (un)substituted sugar, a uronic acid or an oligosaccharide;
R¹ is selected from the group consisting of: 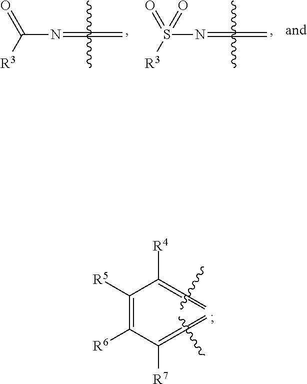
n is selected from the group consisting of: 0, and 1;
Y is selected from the group consisting of: S, O, and C═R²; and
X is selected from the group consisting of: N, N⁺—O⁻, and C—Z;
pharmaceutically acceptable salts, solvates, and physiologically functional derivatives thereof.

7-9. (canceled)

10. A method of inhibiting the LDH-A subunit of an LDH enzyme in mammals which comprises administering to a mammal a therapeutically active amount of a compound selected from the group consisting of:
a compound of formula (I);
a compound of formula (Ia);
a compound of formula (Ib);
a compound of formula (II);
a compound of formula (III); and
a combination thereof.

11. A method of inhibiting LDH5 enzyme in mammals which comprises administering to a mammal a therapeutically active amount of a compound selected from the group consisting of:
a compound of formula (I);
a compound of formula (Ia);
a compound of formula (Ib);
a compound of formula (II);
a compound of formula (III); and
a combination thereof.

12-13. (canceled)

14. A method of treating a condition selected from the group consisting of lymphoma, hepatocellular carcinoma, pancreatic cancer, brain cancer, breast cancer, lung cancer, colon cancer, cervical cancer, prostate cancer, kidney cancer, osteosarcoma, nasopharyngeal cancer, oral cancer, melanoma, ovarian carcinoma; malaria; and idiopathic arthrofibrosis comprising administering an effective amount of a compound of claim 1 to a mammal in need thereof.

15. (canceled)

16. A method of treating a condition selected from the group consisting of lymphoma, hepatocellular carcinoma, pancreatic cancer, brain cancer, breast cancer, lung cancer, colon cancer, cervical cancer, prostate cancer, kidney cancer, osteosarcoma, nasopharyngeal cancer, oral cancer, melanoma, ovarian carcinoma; malaria; and idiopathic arthrofibrosis comprising administering an effective amount of a compound of claim 6 to a mammal in need thereof.
